(19) United States
(12) Patent Application Publication
(10) Pub. No.: US 2024/0122582 A1
(43) Pub. Date: Apr. 18, 2024

(54) ORAL SAMPLING DEVICE

(71) Applicants: University of Washington, Seattle, WA (US); Mahidol University, Nakhon Pathom (TH)

(72) Inventors: Sanitta Thongpang, Seattle, WA (US); Erwin Stefan Peter Berthier, Seattle, WA (US); Damielle Leticia Hieber, Kenmore, WA (US); Xiaojing Su, Seattle, WA (US); Ashleigh Brooks Theberge, Seattle, WA (US); Ulri Nicole Lee, Seattle, WA (US)

(21) Appl. No.: 18/547,125

(22) PCT Filed: Feb. 18, 2022
(86) PCT No.: PCT/US2022/017042

§ 371 Date: Aug. 18, 2023

Related U.S. Application Data
(60) Provisional application No. 63/152,103, filed on Feb. 22, 2021.

(51) Int. Cl. A61B 10/00

(52) U.S. Cl. CPC A61B 10/0051

(57) ABSTRACT

Described herein is an oral sampling device, as well as methods of making and using the oral sampling device. The oral sampling device is configured to be placed inside of a human mouth to capture an analyte(s) found in bodily fluid, such as saliva, produced within the mouth. An example oral sampling device includes a body, at least a portion of which is sized to fit inside of a mouth of a human, has an outer surface, and a recess(es) defined in the outer surface to capture the analyte(s) therein. In some examples, a material of the body within the recess was subjected to a surface treatment to promote the capture of the analyte(s). In some examples, the oral sampling device includes a flavored substance disposed on at least the portion of the body that is to be received inside of a human mouth.

### CROSS-REFERENCE TO RELATED APPLICATION

This application claims the benefit of U.S. Provisional Application No. 63/152,103, titled “ORAL SAMPLING DEVICE” and filed on Feb. 22, 2021, which is incorporated by reference herein in its entirety.

### STATEMENT REGARDING FEDERALLY SPONSORED RESEARCH OR DEVELOPMENT

This invention was made with government support under the National Institutes of Health award number R35GM128648. The government has certain rights in the invention.

### BACKGROUND

Pathogens cause infections in humans, some of which are life-threatening. Acute respiratory infections are common with children, averaging three to five upper respiratory infections per year, some of which may lead to additional complications, if left untreated. In developing countries, illnesses can be even more severe than developed countries, with gaps in healthcare increasing the risk of morbidity and mortality. In order to diagnose and treat certain diseases, a sample of saliva from a human subject can be collected and tested for a specific pathogen.

Traditional sample collection methods used for disease diagnosis include swabbing the posterior pharynx with a pharyngeal swab, extracting sputum, or placing a roll of cotton or an absorbent sponge in the mouth of the human subject to absorb saliva. These sample collection methods are quite unpleasant. For example, swabbing the posterior pharynx or keeping a cotton roll in the mouth for a lengthy period of time can cause discomfort, which makes these collection methods difficult to perform by oneself in a home setting. In addition, visiting a clinic to collect a sample is inconvenient, if not impossible for some people. For example, even in developed countries, residents of rural areas and/or those without health insurance may have limited access to clinics, making it difficult for a portion of the population to receive appropriate diagnosis and treatment. This can discourage adults and children alike from seeking proper testing and treatment. In young children, the aforementioned sample collection methods can cause fear and frustration, resulting in a high rate of non-compliance of sample collection for children. These barriers to adequate sample collection limit the ability to diagnose diseases like strep throat, influenza, tuberculosis, and pneumonia, among others. The disclosure made herein is presented with respect to these and other considerations.

### TECHNICAL FIELD

This disclosure relates to the technical field of devices used for collecting samples of bodily fluid, such as saliva, produced within the oral cavity (mouth) of a human, and methods of making and using such devices.

### SUMMARY

This disclosure provides an oral sampling device, as well as methods of making and using the oral sampling device. The oral sampling device is configured to be placed inside of a human mouth (oral cavity) to capture an analyte(s) found in bodily fluid, such as saliva, produced within the mouth. This disclosure describes various types of analytes that may be captured using the disclosed oral sampling device. In some examples, the oral sampling device is agnostic to the type of analyte(s) that is to be captured by the oral sampling device, meaning that the oral sampling device can be used to capture any type of analyte, and possibly multiple different types of analytes from an individual human subject. In other examples, the oral sampling device is tailored for capturing a specific type of target analyte(s).

An example oral sampling device described herein includes a body. At least a portion of the body is sized to fit inside of a mouth of a human, has an outer surface, and a recess(es) defined in the outer surface. The oral sampling device is an open-fluidic sampling device, meaning that open access to the recess provides a point(s) of ingress for bodily fluid, such as saliva, to enter the recess. Furthermore, the geometry of the recess (e.g., a shape of the recess, a depth of the recess, and/or a width of the recess, etc.) may promote the capture of an analyte(s) found in the bodily fluid (e.g., saliva). For example, the shape of the recess, the depth of the recess, and/or the width of the recess may prevent a tongue from physically scraping the analyte(s) out of the recess without inhibiting entry of the bodily fluid containing the analyte(s) into the recess, thereby mitigating loss of captured analytes. Moreover, increasing (e.g., maximizing) the surface area coverage of the recess(es) on the outer surface of the body can also promote the capture of a greater amount of the analyte(s). Accordingly, in some examples, the recess(es) defined in the outer surface of the body may cover or span at least a threshold percentage of the outer surface. In some examples, at least the portion of the body that is to be received inside of a mouth of a human is made of a rigid porous material in lieu of, or in addition to, having a recess(es) defined in its outer surface. The rigid porous material may allow a bodily fluid, such as saliva, to enter the body of the oral sampling device through pores in the material and may further promote the capture of an analyte(s) found in the bodily fluid (e.g., saliva).

In some examples, a material of the body within the recess(es) was subjected to a surface treatment to promote the capture of an analyte(s) found in bodily fluid, such as saliva. This disclosure describes various types of surface treatment to which the material of the body may have been subjected in order to promote the capture and/or the accumulation of an analyte(s) found in the bodily fluid (e.g., saliva). Example surface treatments include oxygen plasma treatment, coating the material with an affinity reagent(s), and/or hydrophilic treatment, among other surface treatments. The surface treatment may promote the capture and/or the accumulation of the analyte(s) by capturing and/or retaining more of the analyte(s) within the recess(es), as compared to an oral sampling device having a body made of a material that has not been subjected to a surface treatment. In general, the surface treatment may improve the quality of sample collection by ensuring that a sufficient amount of the analyte(s) is/are captured. In some examples, the surface treatment may configure the oral sampling device to capture, and/or accumulate more of, a specific type of target analyte(s).

In some examples, the oral sampling device includes a flavored substance disposed on at least the portion of the body that is to be received inside of a mouth of a human. In some examples, the flavored substance does not cover the recess(es) and/or the flavored substance does not cover at least some of the rigid porous material of the body. This is to allow a bodily fluid (e.g., saliva) containing an analyte(s) to enter the recess(es) and/or the rigid porous material of the body while at least the portion of the body is disposed inside of the mouth of a human subject. The flavored substance is configured to release a flavor over a period of time while at least the portion of the body is disposed inside of the mouth of the human. This period of time may correspond to a target amount of time the oral sampling device is to remain inside of the mouth. Accordingly, the flavored substance may function as a built-in, analog timer by providing a flavor-based indication to the human subject of how long he or she is supposed to keep the oral sampling device in his or her mouth. In other words, the human subject is naturally encouraged to enjoy the flavor while it is released by the flavored substance, and once the flavored substance ceases releasing the flavor, the human subject knows, from the lack of flavor, that it is time to remove the oral sampling device from his or her mouth. The flavored substance may also improve compliance of sample collection in human subjects by making sample collection an enjoyable, fun, and/or appetizing experience, among other benefits. In some examples, the body of the oral sampling device includes a handle and a head coupled to the handle, the head being the portion of the body that is sized to fit inside of a mouth of a human, and a majority of the handle being configured to remain outside of the mouth and grasped by a hand. In this example configuration, the oral sampling device is akin to a lollipop, which is familiar to adults and children alike, and which improves compliance of sample collection. In the case of children, the oral sampling device is a child-friendly device that may reduce anxiety, fear and/or frustration that may otherwise cause a child to resist sample collection. The disclosed oral sampling device also expands the range of diseases that can be detected from a bodily fluid, such as saliva at least because the sensitivity of the diagnostic process is increased by enabling longer accumulation times of the analyte(s).

The present disclosure also provides methods of manufacturing the oral sampling device disclosed herein, as well as methods of using the oral sampling device disclosed herein. In some examples, the manufactured oral sampling device may be packaged and sold to consumers and/or to clinics as a kit for disease diagnosis. Methods of using the oral sampling device include collecting a sample using the oral sampling device and/or extracting a sample (e.g., elution) from the oral sampling device to test the sample for an analyte(s) of interest.

### BRIEF DESCRIPTION OF THE DRAWINGS

FIG. 1A illustrates a close-up, front view of a portion of an example oral sampling device having a spiral groove defined in an outer surface of a head of the oral sampling device.

FIG. 1B illustrates a perspective view of an oral sampling device, a portion of which is depicted in FIG. 1A.

FIG. 1C illustrates a front view of the oral sampling device depicted in FIG. 1B.

FIG. 1D illustrates a side view of the oral sampling device depicted in FIG. 1B.

FIG. 1E illustrates a side, cross-sectional view of the oral sampling device depicted in FIG. 1B.

FIG. 1F illustrates a cross-sectional view of the portion of the oral sampling device depicted in FIG. 1A.

FIG. 2 illustrates analytes being captured within a recess defined in an outer surface of a body of an oral sampling device.

FIG. 3 illustrates a flavored substance of an example oral sampling device dissolving over a period of time during use of the oral sampling device to collect a sample.

FIG. 4 illustrates a close-up, front view of a portion of an example oral sampling device having a radial pattern of one or more grooves defined in an outer surface of a head of the oral sampling device.

FIG. 5 illustrates a close-up, front view of a portion of an example oral sampling device having multiple pits defined in, and spatially distributed across, an outer surface of a head of the oral sampling device.

FIG. 6 illustrates a front view of an example oral sampling device that does not include a handle, a spiral groove being defined in an outer surface of a body of the oral sampling device.

FIG. 7 illustrates an analyte being captured within a recess defined in an outer surface of a body of an oral sampling device, wherein a material of the body within the recess has been surface-treated to promote capture of a specific type of target analyte.

FIG. 8 illustrates a flow diagram of an example process for manufacturing an oral sampling device.

FIG. 9 illustrates a flow diagram of an example process for manufacturing an oral sampling device.

FIG. 10 illustrates a flow diagram of an example process for using an oral sampling device to collect a sample of bodily fluid, such as saliva.

FIG. 11 illustrates a flow diagram of an example process for using an oral sampling device to test a collected sample.

FIG. 12A illustrates that coating a material of the body of the oral sampling device within the recess with fibronectin improves capture of Streptococcus pyogenes.

FIG. 12B illustrates that Streptococcus pyogenes accumulate over time on a fibronectin-coated polystyrene body of the oral sampling device.

FIG. 13 illustrates dissolving periods of flavoring substance of six different types of oral sampling devices.

FIG. 14 illustrates that the geometry of the recess defined in the outer surface of the body of the oral device can mitigate loss of captured analytes in the presence of mechanical friction.

FIG. 15A illustrates images of Streptococcus mutans to demonstrate the efficacy of the elution method described herein.

FIG. 15B illustrates quantitative data to demonstrate the efficacy of the elution method described herein.

### DETAILED DESCRIPTION

As noted above, traditional sample collection methods are unpleasant. With the added inconvenience of visiting a clinic to collect a sample, adults and children alike are discouraged from seeking proper testing and treatment for various diseases using existing sample collection methods. With children, the existing sample collection methods can even cause fear and frustration, resulting in a high rate of non-compliance of sample collection for disease diagnosis. The present disclosure describes, among other things, an oral sampling device that is user-friendly (e.g., pleasant and easy to use), and which can be used at clinics or at home, which improves sample collection compliance and opens the door for telemedicine applications. Also described herein are methods of making and using the oral sampling device. Although several examples described herein pertain to collecting samples of saliva and testing for Streptococcus pyogenes to diagnose group A streptococcal (GAS) pharyngitis (commonly referred to as “strep throat”), it is to be appreciated that the oral sampling device described herein may be used to collect a sample of a bodily fluid (e.g., saliva, mucous, or any other liquid, gas, or viscous material) produced in the oral cavity of a human, and to capture any suitable type of analyte(s) found in the bodily fluid for diagnosing any suitable type of disease(s) or illness(es). Other analyte(s) unrelated to disease and illness can also be captured, for example, for research purposes (e.g., the neuroscience of taste and smell, among other endeavors).

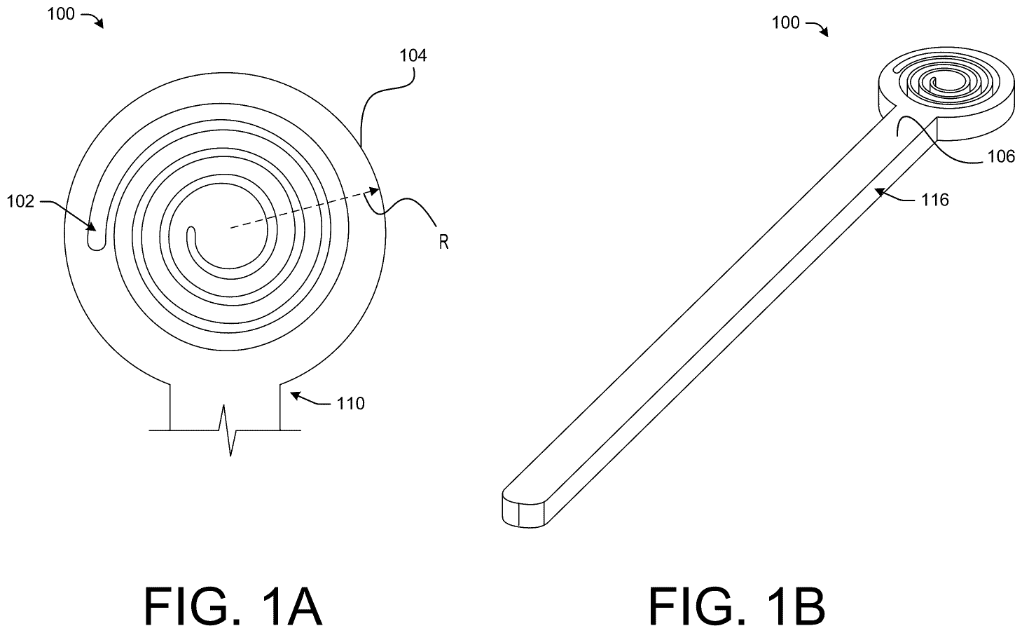

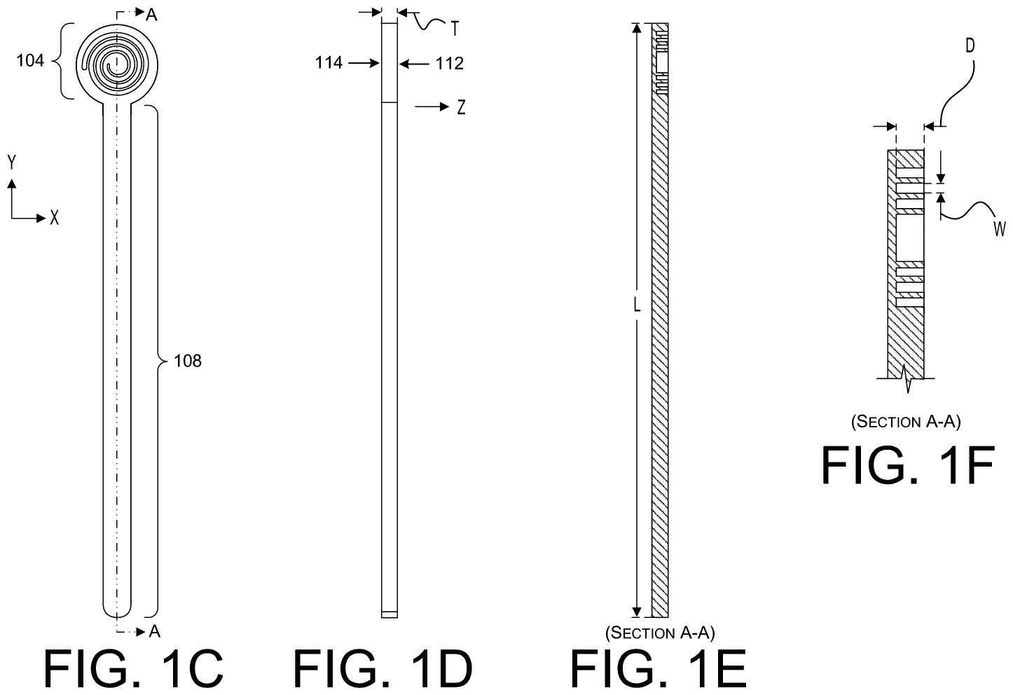

FIG. 1A illustrates a close-up, front view of a portion of an example oral sampling device 100 having a spiral groove 102 defined in an outer surface of a head 104 of the oral sampling device 100. FIGS. 1B, 1C, and 1D illustrate perspective, front, and side views, respectively, of the oral sampling device 100. FIG. 1E illustrates a side, cross-sectional view of the oral sampling device 100 taken along Section A-A shown in FIG. 1C. FIG. 1F illustrates a cross-sectional view of the portion of the oral sampling device 100 depicted in FIG. 1A, which is also taken along Section A-A.

The oral sampling device 100 includes a body 106. The body 106 of the example oral sampling device 100 includes the head 104 and a handle 108 coupled to the head 104 (See e.g., FIG. 1C). For example, the handle 108 may adjoin the head 104 at a neck region 110 (See e.g., FIG. 1A). The terms “troche” component, “lozenge,” or “oral bolus” can be used interchangeably herein with reference to the head 104. The handle 108 is depicted as an elongate member (e.g., a substantially straight stick). However, it is to be appreciated that the handle 108 can be at least partially curved or may have any other suitable shape(s). For example, the handle 108 can alternatively be in the form of a ring or an annular-shaped handle 108 configured to worn on a finger of a hand. The elongate handle 108 shown in FIG. 1C is configured to be grasped by a hand. The handle 108 may prevent a human subject from accidentally swallowing the head 104 when the head 104 is placed inside of the subject's mouth. That is, the handle 108 may improve the safety for human subjects generally, and particularly children, who may be at greater risk of accidentally swallowing objects. Additionally, the handle 108 may allow a human subject to remove (temporarily or permanently) the head 104 from the subject's mouth with ease. When the handle 108 is grasped by a hand of the human subject, the human subject can hold the oral sampling device 100 in his or her hand without setting the oral sampling device 100 on a table or another surface where it may be at risk of contamination, and/or without risk of dropping the oral sampling device 100 on the floor when the head 104 is removed from the subject's mouth. With children in particular, the handle 108 creates a body 106 shaped like a lollipop. This can add to the enjoyment of the experience of collecting a sample, and thereby improve compliance, particularly in children. In other examples described herein, the body 106 does not include a handle 108. As such, it is to be appreciated that the handle 108 is an optional part of the body 106.

The head 104 represents a portion of the body 106 that is configured to be received inside of a mouth of a human subject. For example, the head 104 may be sized to fit inside of a mouth of a human, including a child. The body 106 has one or more outer surfaces. For the example, the body 106 of the oral sampling device 100 includes a front surface 112, a back surface 114, and a side surface 116. Each of these surfaces is an outer surface of the body 106. Because the head 104 and the handle 108 are portions of the body 106, the head 104 and the handle 108 also have these outer surfaces. As shown in FIGS. 1A-1F, the side surface 116 of the body 106 at the top and the bottom of the body 106 can be rounded, and the side surface 116 can extend around an entirety of the body 106. It is to be appreciated, however, that the body 106 may be considered to include a distinct top surface and/or a distinct bottom surface, in some examples, such as if the side surface 116 is planar at the top and/or bottom of the body 106 (e.g., at the distal end of the head 104 and/or at the distal end of the handle 108). The front surface 112 and the back surface 114 of the body 106 can be planar surfaces, and portions of the side surface 116 (e.g., portions along the sides of the handle 108) can also be planar surfaces. Meanwhile, other portions of the side surface 116 (e.g., portions around the head 104 and at the distal end of the handle 108) can be curved surfaces. It is to be appreciated that one or more outer surfaces of the body 106 may be planar or curved, depending on the shape of the body 106. For example, if the body 106 were spherical (e.g., a ball) without a handle, the body 106 may have a single (or continuous) outer surface that is curved.

As noted above, at least a portion of the body 106, such as the head 104, may be sized to fit inside of a mouth of a human, including a child, such as a toddler or even an infant. The head 104 can be disc-shaped and flat, like a coin, as shown in FIGS. 1A-1F. In this example, the head 104 may have a radius, R, of about 5 millimeters (mm). In some examples, the head 104 may have a radius, R, of about 2 mm to 8 mm. In some examples, the size of the head 104 is suitable for inserting the head 104 inside of standard tubes used in clinical chemistry laboratories, such as tubes used in elution kits, such as ESwab 480C (Copan) for Cobas® Liat® (Roche) and ID NOW (Abbott) polymerase chain reaction (PCR) assay analyzer. Although a disc-shaped, flat heat 104 is depicted in FIGS. 1A-1F, it is to be appreciated that other shapes for the head 104 (and/or the body 106) are contemplated herein, such as a spherical head 104, a cuboidal head 104, a pyramidal head 104, a donut shaped head 104, or any other suitable shape. The head 104 (and, in some examples, all of the body 106 or a portion of the body 106) may have a thickness, T, of about 2 mm. In some examples, the head 104 (and/or the body 106) may have a thickness, T, of about 1 mm to 3 mm. The body 106 may have an overall length, L, of about 73 mm when a handle 108 is included. In some examples, the body 106 may have an overall length, L, of about 55 mm to 90 mm when a handle 108 is included.

The spiral groove 102 defined in the outer surface of the head 104 is an example of a recess. Other types of recesses are described herein, and it is to be appreciated that the spiral groove 102 is merely an example of a recess that may be defined in the outer surface of the head 104. Accordingly, it is to be appreciated that any reference to a “recess(es)” herein may include the spiral groove 102 depicted in FIGS. 1A-1F. The recess (e.g., the spiral groove 102) is configured to capture an analyte(s) found in a bodily fluid, such as saliva. Furthermore, the recesses described herein, such as the spiral groove 102, are “open” (as opposed to being enclosed or covered), which provides a point(s) of ingress for the bodily fluid (e.g., saliva), and, hence, the analyte(s) found in the bodily fluid, to enter the recess. The terms “groove” and “channel” may be used interchangeably to describe any type of elongate recess defined in the outer surface of the head 104, such as the spiral groove 102. When the head 104 is placed inside of a human mouth and remains inside of the mouth for a period of time, saliva enters the recess, and an analyte(s) in the saliva may be captured within the recess.

The geometry of the recess may promote the capture and/or the accumulation of an analyte(s) found in a bodily fluid, such as saliva. For example, the cross-sectional shape (e.g., rectangular) of the recess, the depth, D, of the recess, and/or the width, W, of the recess may prevent a tongue from physically scraping the analyte(s) out of the spiral groove 102 without inhibiting entry of the bodily fluid containing the analyte(s) into the recess, thereby mitigating loss of captured analytes. Although the cross-sectional shape of the recesses described herein is shown as being rectangular with sharp corners, other cross-sectional shapes (curved or rounded, without sharp corners) are contemplated. With reference to FIG. 1F, the recess (e.g., the spiral groove 102) may have a depth, D, of about 1.5 mm. In some examples, the recess has a depth, D, of about 1 mm to 2 mm. In some examples, the recess has a depth, D, equal to or greater than 0.5 mm. The recess may have a width, W, of about 0.54 mm. In some examples, the recess has a width, W, of about 0.2 mm to 1 mm. In some examples, the recess has a width, W, equal to or less than 2 mm. This can be specified in terms of the aspect ratio of the cross-sectional area of the recess. As used herein, “aspect ratio” refers to a ratio of a geometric shape's sizes in different dimensions, often expressed as two integer numbers separated by a colon (x:y), less commonly as a simple or decimal fraction. For example, the aspect ratio of the recess—expressed in terms of the width, W, to the depth, D—may be about 0.54:1.5. In some examples, the aspect ratio can be greater than or equal to 1.5 (expressed in terms of the depth, D, to the width, W). In some examples, the aspect ratio can be 2.19, based on a width, W, of 800 micrometers (μm, or microns) and a depth, D, of 1750 μm. In general, the recess may be sufficiently deep and sufficiently thin to prevent the tongue from scraping captured analyte(s) out of the recess. In some examples, the aspect ratio of the recess is selected based on a surface area coverage of the recess (e.g., the spiral grooves 102). In some examples, the aspect ratio of the recess is optimized to have the highest possible surface area for maximizing capture of an analyte(s). In some examples, the recess is a microfluidic groove or channel.

The surface area coverage of the recess(es) may be equal to or greater than a threshold percentage of the surface area of the outer surface of the head 104 in order to capture and/or accumulate more analyte(s). For example, the recess(es) may cover or span at least about 70% of the planar front surface 112 of the head 104. The spiral groove 102, as one example of a recess, may originate from a center of the head 104 and spiral outward from the center. The spiral groove 102 may have at least a threshold number of turns, such as more than one turn, more than two turns, more than three turns, etc. The spiral groove 102 depicted in FIGS. 1A-1F has three turns. In general, increasing (e.g., maximizing) the surface area coverage of the recess(es) defined in the outer surface (e.g., the planar front surface 112) of the body 106 can promote the capture of a greater amount of the analyte(s) found in saliva.

The body 106 can be made of any suitable material(s). In some examples, the body 106 is made of a uniform and/or homogeneous material. In some examples, the body 106 is made of a combination of materials, such as a base material having surface coatings or a homogeneous mixture of two or more materials. The material of the handle 108 may be the same material as the head 104, or it may be a different material. The material of the head 104 (and possibly the handle 108) is a food-safe, nontoxic material. This is because the head 104 is intended to be held inside of the mouth of a human for a period of time while a sample of saliva is collected. The material of the head 104 (and/or the body 106) may be a plastic, a metal, or any other suitable material. In some examples, the material of the head 104 (and/or the body 106) has a pleasant mouth feel, such as a smooth (i.e., non-abrasive) material, and/or rounded edges and/or corners to mitigate injury to the inside of the mouth and/or to improve compliance by creating a pleasant mouth experience during sample collection. In some examples, the material of the head 104 (and/or the body 106), at least within the recess(es), is a polymer (e.g., plastic) material, such as polystyrene. In some examples, the material of the head 104 (and/or the body 106) is a rigid porous material, such as porous polyethylene, porous polypropylene, or any other suitable rigid porous material. An example rigid porous material suitable for the head 104 (and/or the body 106) is Porex®, manufactured by Porex Corporation of Fairburn, Georgia.

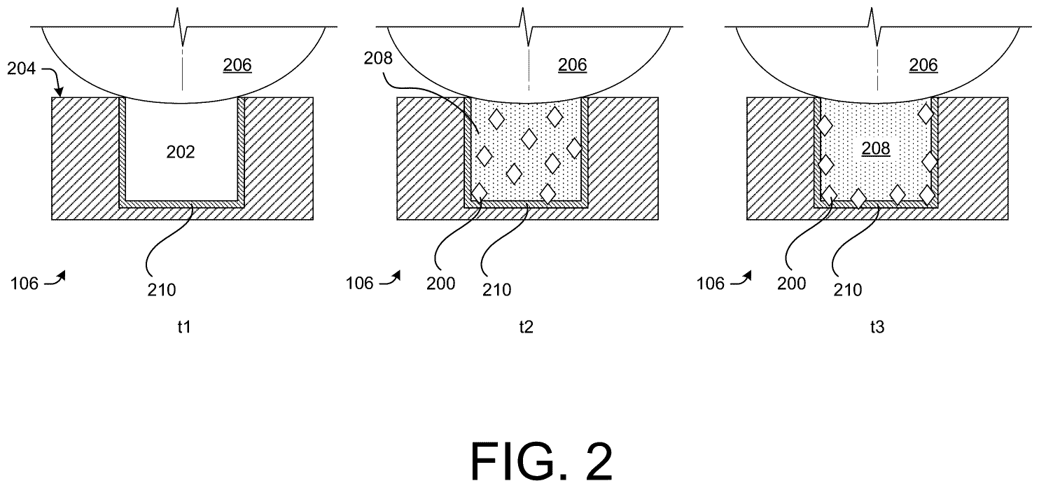

FIG. 2 illustrates analytes 200 being captured within a recess 202 defined in an outer surface 204 of a body 106 of an oral sampling device, such as the oral sampling device 100. The recess 202, in some examples, may represent the spiral groove 102 of the oral sampling device 100, as seen from a cross-sectional view. However, it is to be appreciated that the recess 202 may represent another type of recess, such as those described elsewhere herein. For instance, the recess 202 can be any suitable type of recess including a cavity, a chasm, a channel, a trough, a void, a crevice, a trench, a pit, and/or a groove that is configured to receive and contain a fluid. The outer surface 204, in some examples, may represent the front surface 112 of the body 106 of the oral sampling device 100 described above. The portion of the body 106 depicted in FIG. 2 may represent a portion of the head 104 of the body 106 of the oral sampling device 100, which is configured to be placed inside of a human mouth.

FIG. 2 depicts a tongue 206 in contact with the body 106 and disposed over the recess 202 at three different times, t1, t2, and t3. These times may represent different times over a period of time while at least a portion of the body 106 (e.g., the head 104) is disposed inside of a mouth of a human. Over this period of time, the oral sampling device (e.g., the oral sampling device 100) may be used to capture one or more of the analytes 200 within the recess 202 for evaluation of a condition (e.g., an infection) in the human. For example, at time, t1, the human may have recently inserted a portion of the body 106 (e.g., the head 104) inside of his or her mouth. At time, t2, (after time, t1) saliva 208 has entered the recess 202. Analytes 200 contained in the saliva 208 have also entered the recess 202. At time, t3, (after time, t2) at least some of the analytes 200 have been captured on material 210 of the body 106 within the recess 202.

In some examples, the material 210 of the body 106 within the recess 202 may have been subjected to a surface treatment to promote the capture of the analytes 200 found in the saliva 208. Although the material 210 within the recess 202 is depicted in FIG. 2 as being distinct from a remainder of the body 106, this is meant to represent that the material of the body 106 within the recess 202 has been subjected to a surface treatment. It is to be appreciated that other surfaces of the body 106 (e.g., surfaces outside of the recess 202) may be subjected to the surface treatment, such as by surface-treating the entire body. The surface treatment of the material 210 may promote the capture and/or the accumulation of the analytes 200 by capturing and/or retaining more of the analytes 200 within the recess 202. In some examples, the surface treatment to which the material 210 has been subjected may configure the oral sampling device (e.g., the oral sampling device 100) to capture, and/or accumulate more of, a specific type of target analyte(s). Accordingly, the analytes 200 depicted in FIG. 2 may represent a specific type of target analyte, in some examples. In some examples, the analytes 200 depicted in FIG. 2 may represent the same type of analyte or different types of analytes that are captured using an oral sampling device that is agnostic to the type of analyte(s) to be captured.

In some examples, the surface treatment may include oxygen plasma treatment. In this example, the material 210 may be a plastic, such as polystyrene. In order to surface-treat the material 210, the body 106 of the oral sampling device may be inserted into an oxygen plasma treatment apparatus, and treated using the oxygen plasma treatment. This causes covalent changes in the material 210 where hydrophilic groups are appended to the surface. With polystyrene material, the oxygen plasma treatment facilitates cell adhesion on the surface of the material. After oxygen plasma treatment, the material 210 is configured to attract and retain analytes, such as the analytes 200. This type of surface treatment is agnostic to the type of analyte(s) to be captured, which means that the surface-treated material 210 of the body 106 within the recess 202 may attract multiple different types of analytes, and possibly other particles that are not of interest for disease diagnosis.

In some examples, the surface treatment may include coating a material of the body 106 within the recess 202 with an affinity reagent(s). Accordingly, the material 210 depicted in FIG. 2 may represent such a coating. In some examples, the affinity reagent may be an aptamer, a protein, such as fibronectin or collagen, or any other suitable molecule. Methods of making aptamers are known in the art, and described, for example, in Huang et al. Anal. Chem., 2008, 80 (3), pp 567-572. Protein refers to any of various naturally occurring substances that include amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies). Fibronectin refers to a high-molecular weight glycoprotein of the extracellular matrix that binds to membrane-spanning receptor proteins called integrins. In some examples, the affinity reagent may be a hydrogel, a fiber matrix, or a matrix protein. In some examples, coating the material of the body 106 within the recess 202 with an affinity reagent(s) may functionalize the material (e.g., the material 210 shown in FIG. 2) through surface chemistry modifications, such as by introducing a functional group into a molecule of the body 106 material to alter the chemical behavior of the molecule, such as by attaching molecules or nanoparticles to the surface of the body 106 material within the recess 202. In some examples, the affinity reagent is a binding molecule for the analyte 200. In some examples, the binding molecule for the analyte 200 is a nucleic acid that hybridizes to the analyte 200 or is a protein binding domain that binds the analyte 200. In some examples, the protein binding domain is an antibody or a binding fragment thereof. In some examples, the binding fragment thereof is a single chain variable fragment (scFv).

Protein Binding Domains. In certain examples, the current disclosure provides protein binding domains to capture a protein analyte of interest. In particular embodiments, protein binding domains include cell marker ligands, receptor ligands, antibodies, peptides, peptide aptamers, nucleic acids, nucleic acid aptamers, spiegelmers or combinations thereof. Within the context of the disclosure, protein binding domains include any substance that binds to another substance to form a complex capable of supporting detection in an analyte assay. Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR). The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); North et al. (2011) J. Mol. Biol. 406(2):228-56 (North numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. North numbering uses longer sequences in the structural analysis of the conformations of CDR loops. CDR residues within a given antibody can also be identified using software programs such as ABodyBuilder.

In some instances, protein binding domains include scFvs. scFvs can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to binding to protein analytes.

Other binding fragments, such as Fv, Fab, Fab′, F(ab′)2, and VH domain antibodies can also be used. VH domain antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

Functional variants of protein binding domains include one or more residue additions or substitutions that do not substantially impact the physiological effects of the protein binding domains. Functional fragments include one or more deletions or truncations that do not substantially impact the physiological effects of the protein binding domains. A lack of substantial impact can be confirmed by observing experimentally comparable binding. Functional variants and functional fragments of protein binding domains bind their cognate antigen or ligand at a level comparable to a wild-type reference.

In particular embodiments, a VL region in a protein binding domain of the present disclosure is derived from or based on a VL of a known antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of a known antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the known antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a protein binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, of a known antibody wherein each CDR includes zero changes or at most one, two, or three changes, from an antibody disclosed herein or fragment or derivative thereof that specifically binds an analyte of interest.

Particular embodiments to capture analytes can utilize binding domains from anti-RSV antibodies, anti-HIV antibodies, anti-Dengue virus antibodies, anti-Bordatella pertussis antibodies, anti-hepatitis C antibodies, anti-influenza virus antibodies, anti-parainfluenza virus antibodies, anti-metapneumovirus (MPV) antibodies, anti-cytomegalovirus antibodies, anti-Epstein Barr virus antibodies; anti-herpes simplex virus antibodies, anti-Clostridium difficile bacterial toxin antibodies, or anti-tumor necrosis factor (TNF) antibodies. Known anti-RSV antibodies include palivizumab; those described in U.S. Pat. No. 9,403,900; AB1128 (available from MILLIPORE) and ab20745 (available from ABCAM). An example of a known anti-HIV antibody is 10E8, which is a broadly neutralizing antibody that binds to gp41. VRC01, which is a broadly neutralizing antibody that binds to the CD4 binding site of gp120. Other exemplary anti-HIV antibodies include ab18633 and 39/5.4A (available from ABCAM); and H81E (available from THERMOFISHER). Examples of an anti-Dengue virus antibodies include antibody 55 (described in U.S. 20170233460); antibody DB2-3 (described in U.S. Pat. No. 8,637,035); and ab155042 and ab80914 (both available from ABCAM). An anti-pertussis antibody is described in U.S. Pat. No. 9,512,204. Examples of anti-hepatitis C antibodies include MAB8694 (available from MILLIPORE) and C7-50 (available from ABCAM). Anti-influenza virus antibodies are described U.S. Pat. No. 9,469,685 and also include C102 (available from THERMOFISHER). An exemplary anti-MPV antibody includes MPE8. Exemplary anti-CMV antibodies includes MCMV5322A, MCMV3068A, LJP538, and LJP539. See also, for example, Deng et al., Antimicrobial Agents and Chemotherapy 62(2) e01108-17 (February 2018); and Dole et al., Antimicrobial Agents and Chemotherapy 60(5) 2881-2887 (May 2016). Examples of anti-HSV antiboiesy include HSV8-N and MB66. Exemplary anti-Clostridium difficile antibodies include actoxumab and bezlotoxumab. See also, for example, Wilcox et al., N Engl J Med 376(4) 305-317 (2017). Numerous additional antibody sequences are available and known to those of ordinary skill in the art that can be used within the teachings of the current disclosure. Sequence information for commercially available antibodies may be found in the Drug Bank database, the CAS Registry, and/or the RSCB Protein Data Bank.

In certain examples, nucleic acids that hybridize have complete sequence complementarity that results in hybridization, however complete sequence complementarity is not always required. Hybridization can also be assessed, for example, under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

When utilizing commercially available protein sequences (e.g., antibody sequences) as indicated previously, variants can be used. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The use of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Variants of the proteins and nucleic acids disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to a reference sequence.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

In certain examples, a protein binding domain “specifically binds” its analyte. Specifically binds refers to an association of a protein binding domain to its cognate analyte with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 1⁰⁷ M-1, at least 10⁸ M-1, at least 109 M-1, at least 10¹⁰ M-1, at least 10¹¹ M-1, at least 10¹² M-1, or at least 10¹³ M-1. In particular embodiments, “low affinity” binding domains refer to those binding domains with a Ka of up to 1⁰⁷ M-1, up to 10⁶ M-1, up to 105 M-1. Alternatively, affinity may be defined as an equilibrium dissociation constant (Kd) of a particular binding interaction with units of M (e.g., 10⁻⁵ M to 10⁻¹³ M). In certain embodiments, a protein binding domain may have “enhanced affinity,” which refers to a selected or engineered binding domain with stronger binding to a cognate binding molecule than a wild type (or parent) protein binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of aspects of the present disclosure can employ techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

In some examples, the surface treatment may include hydrophilic treatment. In this example, the hydrophilic treatment may involve placing the body 106 of the oral sampling device into an airtight chamber, lowering the pressure within the chamber using a vacuum (e.g., removing substantially all of the air within the chamber), and inserting a precursor gas into the low-pressure chamber. The gas may ionize under these conditions and become plasma, and the excited plasma ions may collide with the surface of the body, microscopically changing the geometry of the surface. In some examples, the surface of the material 210 of the body 106 undergoes oxidation and the bombarding plasma ions form hydroxyl groups on the surface, which are polar. This may cause saliva (which is water-based) to be attracted to the hydroxyl groups, thereby promoting saliva exchange within the recess 202 by enhancing the adhesion of saliva and the analytes contained therein to the surface of the material 210 of the body 106 within the recess 202.

The analytes 200 captured by the oral sampling device 100 may be any suitable type of analyte. In some examples, the analytes 200 represent a pathogen, such as a bacteria, a virus, or the like. In some examples, the analytes 200 represent Streptococcus pyogenes. In some examples, the analytes 200 represent a virus within the family Rhabdoviridae, Arenaviridae, Togaviridae, Filoviridae, Retroviridae, Coronaviridae, Paramyxoviridae, Flaviviridae, Orthomyxoviridae, and Baculoviridae. In particular embodiments, the analytes 200 represent proteins, such as glycoproteins (GPs) derived from a genus including Vesiculovirus, Lyssavirus, Arenavirus, Alphavirus, Filovirus, Alpharetrovirus, Betaretrovirus, Gammaretrovirus, Deltaretrovirus, Spumavirus, Lentivirus, Coronavirus, Respirovirus, Hepacivirus, Influenzavirus A, or nucleopolyhedrovirus. In some examples, the analytes 200 represent a virus such as coronavirus, respiratory syncytial virus (RSV), influenza virus, parainfluenza virus hepatitis virus, human immunodeficiency virus (HIV), measles virus, Dengue virus, Epstein Barr virus, or herpes simplex virus. In some examples, the coronavirus is Middle East respiratory syndrome coronavirus (MERS-CoV), severe acute respiratory syndrome coronavirus (SARS-CoV), or severe acute respiratory syndrome coronavirus 2 (SARS-CoV-2). In some examples, the analytes 200 represent a human-associated biomarker, including biomarkers that are not related to a pathogen disease. In some examples, the analytes 200 may represent a molecule (e.g., oral biomolecule) or a particle that can be obtained through (or found in) saliva, including those found in saliva at low concentrations or trace amounts. In some examples, the analyte 200 may represent a nucleic acid (e.g., deoxyribonucleic acid (DNA), ribonucleic acid (RNA), etc.).

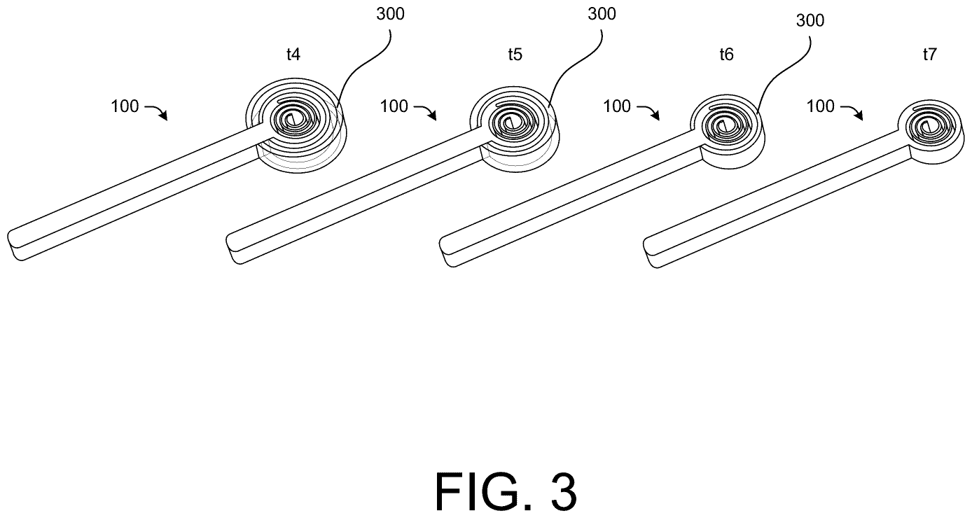

In some examples, the oral sampling device 100 may include a flavored substance 300 disposed on at least a portion of the body 106, such as the head 104. FIG. 3 illustrates the flavored substance 300 disposed on the head 104 of an example oral sampling device 100 dissolving over a period of time during use of the oral sampling device 100 to collect a sample. In certain examples, the flavored substance 300 does not cover the recess 202 (e.g., the spiral groove 102) or does not cover the entirety of the recess. For example, the flavored substance 300 may be disposed on the side surface 116 of the body 106 (e.g., at the head 104), and/or the back surface 114 of the body 106 (e.g., at the head 104), and/or a portion of the front surface 112 of the body 106, such as at the periphery of the front surface 112 such that the recess 202 (e.g., the spiral groove 102) remains exposed/uncovered to provide a point(s) of ingress for saliva 208 containing an analyte(s) 200 to enter the recess 202. In some examples, the flavored substance 300 is not disposed on the front surface 112 of the body 106, but is disposed on the side surface 116 and/or the back surface 114 of the body 106 (e.g., at the head 104). In certain examples, the flavored substance may cover the recess, or portions thereof, in a manner configured to dissipate from the covered area rapidly upon initiation of use (e.g., introduction into the oral cavity of a human).

In certain examples, the flavored substance 300 is configured to release (e.g., steadily release) a flavor over a period of time while at least a portion of the body 106 (e.g., the head 104) is disposed inside of a mouth of a human. This period of time may substantially correspond to a target amount of time the oral sampling device 100 is to remain inside of the mouth. Accordingly, the flavored substance 300 may function as a built-in, analog timer to provide a flavor-based indication to the human subject of how long he or she is supposed to keep the head 104 of the oral sampling device 100 in his or her mouth. In other words, the human subject is naturally encouraged to enjoy the flavor while it is released, and once the flavored substance 300 ceases releasing the flavor (e.g., after the flavored substance 300 is dissolved), the human subject knows that it is time to remove the oral sampling device 100 from his or her mouth. In the example of FIG. 3, the flavored substance 300 may be a hard candy or candy substitute, such as an artificial sweetener (e.g., flavored isomalt), which is configured to releases a flavor (e.g., fruit flavor) and dissolve over the period of time while at least the portion of the body 106 (e.g., the head 104) is disposed inside of the mouth of the human. The release of flavor by the flavored substance 300 may be triggered by moisture or hydration (e.g., saliva contact). As noted above, the flavored substance 300 may improve compliance of sample collection in human subjects by making sample collection an enjoyable, fun, and/or appetizing experience, among other benefits. The example oral sampling device 100 with the flavored substance 300 shown in FIG. 3 is akin to a lollipop, which is familiar to adults and children alike to improve compliance of sample collection, and in the case of children, such an oral sampling device may reduce anxiety, fear and/or frustration associated with sample collection.

To illustrate the built-in, analog timer aspect of the flavored substance 300, FIG. 3 illustrates how the flavored substance 300 changes over the period of time while a portion of the body 106 (e.g., the head 104) of the oral sampling device 100 is inside of the mouth of the human subject. For example, at time, t4, the human may have recently inserted a portion of the body 106 (e.g., the head 104) with the flavored substance 300 inside of his or her mouth. At time, t5, (after time, t4) the flavored substance 300 has partially dissolved, causing the flavored substance 300 to decrease in size. Meanwhile, analytes 200 are being captured within the recess 202 (e.g., the spiral groove 102) between times t4 and t5. At time, t6, (after time, t5) the flavored substance 300 has further dissolved, causing the flavored substance 300 to further decease in size. Meanwhile, between times t5 and t6, at least some of the previously captured analytes 200 are retained within the recess 202 (e.g., the spiral groove 102) and additional analytes 200 are accumulated within the recess 202 (e.g., the spiral groove 102) to increase the concentration of the analytes 200 in the collected sample. At time, t7, (after time, t6) the flavored substance 300 is completely dissolved and ceases releasing flavor as a result of dissolving. At time, t7, the human may remove the oral sampling device 100 from his or her mouth to complete the sample collection process, whereby the captured analytes 200 that remain within the recess 202 (e.g., the spiral groove 102) can be used to evaluate a condition (e.g., an infection) of the human subject, such as to diagnose a respiratory disease, like pharyngitis. The human subject knows that it is time to remove the oral sampling device 100 from his or her mouth at time, t7, due to the cessation of flavor release by the flavored substance 300.

The flavored substance 300 may be hard (e.g., a hard candy) or soft (e.g., a gel, a gummy material having a “gummy” consistency). A “gel,” as used herein, is a semi-solid that exhibits plastic deformation under applied force. A “gummy material,” as used herein, is a gelatin-based substance that exhibits elastic deformation under applied force. The flavored substance 300 may be a candy (e.g., a sugar-based candy) or a candy substitute, such as isomalt with flavoring. Isomalt (e.g., sugar alcohol isomalt) is relatively easy to mold, resists crystallization, and has a relatively long shelf life. Isomalt has a glass transition state of 63.6 degrees Celsius (° C.), which makes isomalt stable in solid form. Isomalt also has a low hygroscopicity (ERH of 88), meaning it will not absorb water until equilibrium relative humidity reaches 88% at 20° C., which enables the isomalt to remain stable if packaged and stored for extended periods of time. Additionally, in the presence of oral bacteria, such as Streptococcus mutans, sucrose ferments and becomes turbid while isomalt and other polyols do not. Isomalt also has a glycemic index of two and is safe for diabetic patients. To prevent human subjects from “crunching” the flavored substance 300, a plastic backing may be disposed on the flavored substance 300 at the back surface 114 of the body 106.

In some examples, the amount of flavored substance 300 that is disposed on at least a portion (e.g., the head 104) of the body 106 is selected to control the amount of time the portion (e.g., the head 104) of the body 106 spends inside of the mouth of a human subject (sometimes referred to herein as the “sample collection period”). The amount of the flavored substance 300 can be defined in various ways, such as the thickness of the flavored substance 300 measured in the Z-direction (See FIG. 1D), the height of the flavored substance 300 that extends above the outer surface of the head 104, the mass of the flavored substance 300, the surface area-to-mass ratio of the flavored substance 300, and/or the surface area-to-volume ratio of the flavored substance 300. By varying the amount of the flavored substance 300 via any of these metrics, the rate at which the flavored substance 300 releases flavor and/or the duration over which the flavor is released can be adjusted. By lengthening the sample collection period using a selected amount of flavored substance 300, analytes 200 may accumulate within the recess 202 (e.g., the spiral groove 102) at higher concentrations, regardless of whether the analyte 200 is abundant or in trace amounts within saliva. In some examples, the sample collection period can be tailored using a selected amount of flavored substance 300 to accumulate more of the analytes, and/or to capture analytes 200 found in saliva in trace amounts that nevertheless accumulate within the recess 202 (e.g., the spiral groove 102) in higher concentrations over the sample collection period. This can allow for capturing biomarkers that are not considered to be “saliva” biomarkers because they are often found in trace amounts in saliva. In some examples, the flavored substance 300 may stimulate saliva generation, thereby increasing the amount and/or flow of saliva within the mouth to help accumulate higher concentrations of the analyte 200. In some examples, the flavored substance 300 is configured to stimulate a neurological response that causes the human subject to produce an analyte 200 found in saliva so that the analyte 200 can be captured during the sample collection period. In this example, the analyte 200 may represent a biomarker associated with a neurological response related to taste.

As mentioned above, the flavored substance 300 may be configured to release a flavor over a limited period of time by dissolving over the period of time. In some examples, the flavored substance 300 is configured to release a flavor without dissolving or otherwise disappearing. For example, the flavored substance 300 may be a gel that is configured to release a flavor without dissolving in the mouth of a human subject. In some examples, the flavored substance 300 is configured to change color after a portion (e.g., the head 104) of the body 106 has been disposed inside of the mouth for the period of time corresponding to the sample collection period. For example, the flavored substance 300 may be configured to change from red to blue when the oral sampling device 100 has remained in the mouth for the period of time corresponding to the sample collection period, which may coincide with the cessation of flavor release. The color change of the flavored substance 300 is another example indicia that can indicate to the human subject that it is time to remove the oral sampling device 100 from the mouth.

Beyond hard candy, gel, and gummy materials, other forms may also be utilized. For example, exemplary flavored substances that may be used for the flavored substance 300 include capsules, coated tablets, gelcaps, granules, gums, pastes, pellets, rapidly-dissolving tablets, sachets, semi-solids, etc.

In some examples, the flavored substance 300 includes one or more excipients. Exemplary excipient classes include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

One or more colorants may be included in the flavored substance 300 to impart color to the formulation. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

One or more diluents may be included in the flavored substance 300 to enhance the granulation of the flavored substance 300. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin.

One or more disintegrants may be included in the flavored substance 300 in order to facilitate dissolution. Disintegrants, including permeabilising and wicking agents, are capable of drawing water or saliva up into the flavored substance 300 which promotes dissolution from the inside as well as the outside of the flavored substance 300. Such disintegrants, permeabilising and/or wicking agents that may be used include starches, such as corn starch, potato starch, pre-gelatinized and modified starches thereof, cellulosic agents, such as Ac-di-sol, montmorrilonite clays, cross-linked polyvinylpyrrolidone (PVP), sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose. Dissolution of the flavored substance 300 may be facilitated by including relatively small particles sizes of the ingredients used.

The flavored substance 300 may include one or more dispersing or suspending agents. Exemplary dispersing or suspending agents include acacia, alginate, dextran, fragacanth, gelatin, hydrogenated edible fats, methylcellulose, PVP, sodium carboxymethyl cellulose, sorbitol syrup, and synthetic natural gums.

The flavored substance 300 may include one or more emulsifiers. Exemplary emulsifiers include acacia and lecithin.

The flavor that is to be released by the flavored substance 300 may be due to the flavored substance 300 including a flavorant(s), such as one or more natural or artificial compounds used to impart a pleasant flavor and often odor to flavored substance 300. Exemplary flavorants include, natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that may be used include natural berry extracts and natural mixed berry flavor, as well as citric and malic acid.

The flavored substance 300 may include one or more glidants to improve the flow of powder blends during manufacturing and minimize weight variation of the flavored substance 300. Exemplary glidants include silicon dioxide, colloidal or fumed silica, magnesium stearate, calcium stearate, stearic acid, cornstarch, and talc.

The flavored substance 300 may include one or more lubricants. Lubricants are substances that reduce friction during composition compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

The flavored substance 300 may include one or more preservatives. Exemplary preservatives include methyl p-hydroxybenzoates, propyl p-hydroxybenzoates, and sorbic acid.

The flavored substance 300 may include one or more sweeteners. Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

The flavored substance 300 may include sugars to improve mouthfeel and palatability. Exemplary sugars include white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof. Fondant or gums such as gelatin, agar, arabic gum, guar gum, and carrageenan may be added, in some examples. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° C. to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of the flavored substance 300. By “enhance the texture” it is meant that the alkyl polysiloxane improves one or more of the stiffness, the brittleness, and the chewiness of the flavored substance 300, relative to the same preparation lacking the alkyl polysiloxane. By “enhance the mouthfeel” it is meant that the alkyl polysiloxane reduces the gritty texture of the flavored substance 300 once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the back bone. Depending upon their grade, they can further include silica gel. Alkyl polysiloxanes are generally viscous oils. Exemplary alkyl polysiloxanes that can be used in the flavored substance 300 include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than 90.5% by weight (CH₃)₃—Si{OSi(CH₃)₂}CH₃ in admixture with 4.0% to 7.0% by weight SiO₂.

To prevent the stickiness and to facilitate conversion of the active ingredients to emulsion or suspension upon taking, the flavored substance 300 may further include emulsifiers such as glycerin fatty acid ester, sorbitan monostearate, sucrose fatty acid ester, lecithin and mixtures thereof. In particular embodiments, one or more of such emulsifiers may be present in an amount of 0.01% to 5.0%, by weight of the administered formulations. If the level of emulsifier is lower or higher, in particular embodiments, an emulsification cannot be realized, or wax value will rise.

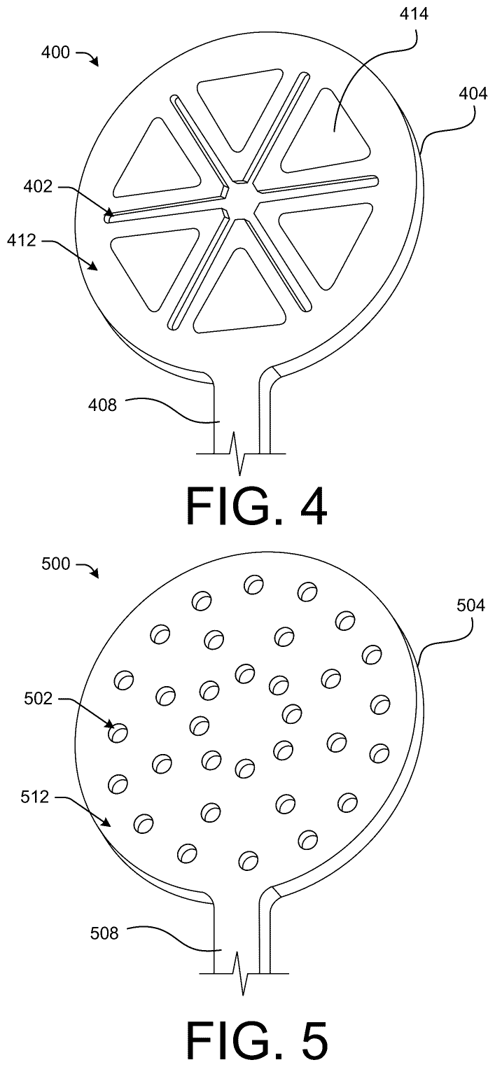

FIG. 4 illustrates a close-up, front view of a portion of an example oral sampling device 400 having a radial pattern of one or more grooves 402 defined in an outer surface of a head 404 of the oral sampling device 400. In FIG. 4, the head 404 includes a front surface 412, which is an outer surface of the head 404. The oral sampling device 400 may further include a handle 408 coupled to the head 404. The head 404 and the handle 408 are portions of a body of the oral sampling device 400, which may be similar to the body 106 of the oral sampling device 100 described earlier. In FIG. 4, the head 404 is disc-shaped and flat, like a coin, and the front surface 412 is a planar surface. The one or more grooves 402 are defined in the planar front surface 412 of the head 404. The radial pattern of the one or more grooves 402 is such that a number of radially-oriented, elongate grooves 402 extend from a center of the head 404 toward a periphery of the head 404. In FIG. 4, there are six radially-oriented, elongate grooves 402 defined in the planar front surface 412 of the head 404, the grooves 402 being circumferentially spaced apart, like spokes of a wheel. This radial pattern is sometimes referred to herein as a “star” pattern, due to its resemblance of a star shape.

Like the spiral groove 102 of the oral sampling device 100, the groove(s) 402 of the oral sampling device 400 is an example of a recess 202. The groove(s) 402 is also an open groove to provide a point(s) of ingress for the bodily fluid (e.g., saliva), and, hence, the analyte(s) found in the bodily fluid, to enter the groove(s) 402. For example, when the head 404 is placed inside of a human mouth and remains inside of the mouth for a period of time, saliva 208 enters the groove(s) 402 and an analyte(s) 200 in the saliva 208 may be captured in the groove(s) 402. In some examples, there may be at least a threshold number of radially-oriented, elongate grooves 402 defined in the planar front surface 412 of the head 404 to increase the surface area coverage of the groove(s) 402, thereby promoting the capture of a greater amount of the analyte(s) found in saliva.

In some examples, a flavored substance 414 is disposed on at least a portion of the oral sampling device's 400 body, such as the head 404. For example, the flavored substance 414 may be disposed within recessed areas defined in the planar front surface 412 of the head 404 to form one or more “gel pads” on the planar front surface 412 of the head 404. In FIG. 4, these recessed areas are interposed between pairs of adjacent elongate grooves 402 that extend radially from the center of the head 404, and they are triangular in shape. Accordingly, the groove(s) 402 and the flavored substance 414 can be arranged in a manner that resembles a cross-section of a citrus fruit, with the flavored substance 414 being disposed in triangular-shaped recessed areas defined in the front surface 412 of the head 402.

The flavored substance 414 may be similar to the flavored substance 300 described above. For example, the flavored substance 414 is configured to release (e.g., steadily release) a flavor over a period of time while at least a portion of the body (e.g., the head 404) is disposed inside of a mouth of a human. In an example, the flavored substance 414 may be a gel that is configured to release a flavor over a period of time while saliva containing an analyte(s) 200 is captured within the groove(s) 402.

FIG. 5 illustrates a close-up, front view of a portion of an example oral sampling device 500 having multiple pits 502 (sometimes referred to as “voids 502) defined in, and spatially distributed across, an outer surface of a head 504 of the oral sampling device 500. In FIG. 5, the head 504 includes a front surface 512, which is an outer surface of the head 504. The oral sampling device 500 may further include a handle 508 coupled to the head 504. The head 504 and the handle 508 are portions of a body of the oral sampling device 500, which may be similar to the body 106 of the oral sampling device 100 described earlier. In FIG. 5, the head 504 is disc-shaped and flat, like a coin, and the front surface 512 is a planar surface. The multiple pits 502 are defined in the planar front surface 512 of the head 504.

Like the spiral groove 102 of the oral sampling device 100, the pits 502 of the oral sampling device 500 are each an example of a recess 202. Each pit 502 is also an open pit to provide a point(s) of ingress for the bodily fluid (e.g., saliva), and, hence, the analyte(s) found in the bodily fluid, to enter the pit 502. For example, when the head 504 is placed inside of a human mouth and remains inside of the mouth for a period of time, saliva 208 enters the pits 502 and an analyte(s) 200 in the saliva 208 may be captured in the pits 502. In some examples, there may be at least a threshold number of pits 502 defined in the planar front surface 512 of the head 504 to increase the surface area coverage of the pits 502, thereby promoting the capture of a greater amount of the analyte(s) found in saliva. The array of pits 502 may be uniformly spaced, non-uniformly spaced, symmetric, or asymmetric. In some examples, the array of pits 502 are spatially distributed across the outer surface (e.g., planar front surface 512) of the head 504 in a pattern (e.g., a spiral pattern).

In some examples, a flavored substance, such as the flavored substance 300, may be disposed on at least a portion of the oral sampling device's 500 body, such as the head 504. In such an implementation, the flavored substance does not cover the pits 502 to allow a bodily fluid, such as saliva, to enter the pits 502 while the head 504 is disposed in the mouth of a human subject.

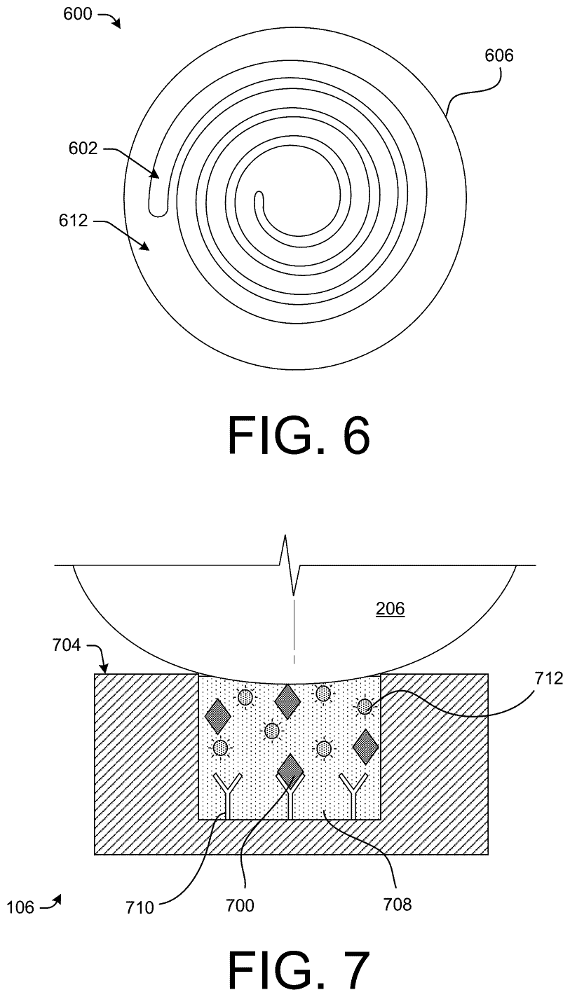

FIG. 6 illustrates a front view of an example oral sampling device 600 having a spiral groove 602 defined in an outer surface of a body 606 of the oral sampling device 600. In FIG. 6, the body 606 of the oral sampling device 600 does not include a handle. In FIG. 6, the body 606 includes a front surface 612, which is an outer surface of the body 606. In FIG. 6, the body 606 is disc-shaped and flat, like a coin, and the front surface 612 is a planar surface. The spiral groove 602 is exemplary, and it is to be appreciated that the body 606 can have one or more recesses 202 defined in an outer surface thereof, as described in the various other examples herein, such as the radial pattern of one or more grooves 402, the array of multiple pits 502, or any other pattern and/or shape of recess(es). Any of the oral sampling devices described herein may have a groove(s) defined in an outer surface of the body of the oral sampling device, the groove(s) having a grid pattern, an angular pattern, or any suitable pattern. In some examples, the groove(s) defined in the outer surface of the body may depict a face of a character (e.g., a familiar cartoon character from a movie or a show). Accordingly, while example patterns and shapes of recesses 202 have been described herein, it is to be appreciated that a recess(es) of any shape and/or pattern can be defined in an outer surface of a body of an oral sampling device to capture analytes found in a bodily fluid, such as saliva, the recess(es) 202 having any suitable polygonal shape of cross-section and/or aperture (e.g., the pits 502 may be square or triangular in shape, rather than circular), in any suitable pattern that is symmetric, asymmetric, uniform, non-uniform or the like.

FIG. 7 illustrates a specific type of target analyte 700 being captured within a recess (e.g., the recess 202 of FIG. 2), the recess defined in an outer surface 704 of a body 106 of an oral sampling device. In FIG. 7, a material of the body 106 within the recess has been subjected to surface treatment to promote capture of the analyte 700. FIG. 7 depicts a tongue 206 in contact with the body 106 and disposed over the recess, such as when at least a portion of the body 106 (e.g., the head) is disposed inside of a mouth of a human. In FIG. 7, a specific affinity reagent(s) can be used to surface-treat the material of the body 106 within the recess to selectively capture an analyte 700 of interest, such as a target pathogen. The surface treatment may include coating the material of the body 106 (at least within the recess) with the specific affinity reagent(s), where the affinity reagent(s) can be any of the types of affinity reagents described herein. In FIG. 7, the surface-treated material 710 of the body 106 may attract and capture the analyte 700 of interest while rejecting other particles 712 (e.g., bacteria that is not of interest for disease diagnosis) found in the saliva 708 that has entered the recess. This technique of selectively capturing a specific type of target analyte(s) 700 can make testing the sample to evaluate a condition (e.g., an infection) of the human subject easier. In some examples, this example technique can create an oral sampling device (e.g., the oral sampling device 100) that is specifically tailored for a particular diagnostic purpose (e.g., to capture a target type of analyte 700).

The processes described herein are illustrated as a collection of blocks in a logical flow graph, which represent a sequence of operations. The order in which the operations are described is not intended to be construed as a limitation, and any number of the described blocks can be combined in any order and/or in parallel to implement the processes.

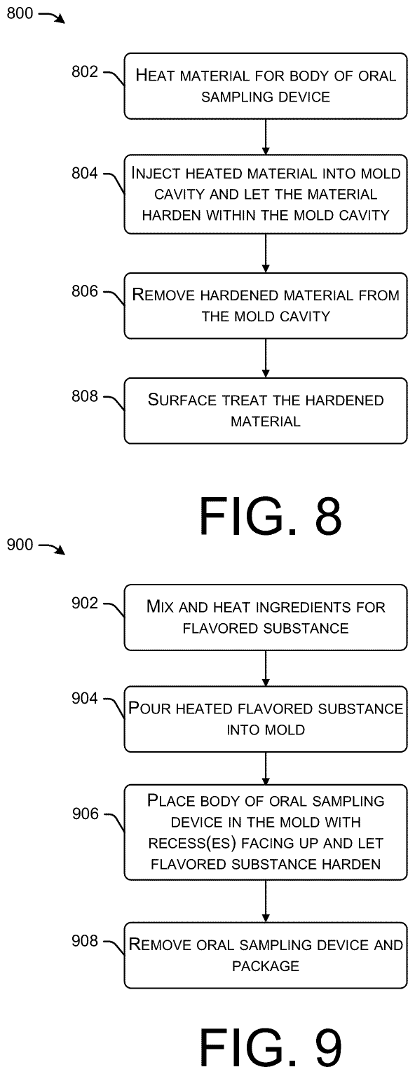

FIG. 8 illustrates a flow diagram of an example process 800 for manufacturing an oral sampling device. For discussion purposes, the process 800 is described with reference to the previous figures.

At 802, a material that is to be used for the body of an oral sampling device (e.g., the body 106 of the oral sampling device 100) is heated to an elevated temperature at which the material melts into a liquid form. The temperature to which the material is heated may vary depending on the material. In an example, the material is polystyrene and the heating at block 802 includes heating the polystyrene to a temperature at or above the melting point of polystyrene (e.g., 240° C.). The heating at block 802 may be performed while the material is within a container (e.g., a mixing barrel). The container may use a mixing mechanism (e.g., a helical screw) to mix the heated material, in some examples.

At 804, the heated material is injected into a mold cavity as part of an injection molding procedure. The material is left in the mold cavity for a period of time to cool and harden. Accordingly, at block 804, the temperature of the material may gradually decrease to a temperature below the melting point of the material such that the material solidifies. The mold cavity may have a shape that corresponds to the body of the oral sampling device, and, in some examples, the mold cavity may include a protrusion(s) that is used to form the recess(es) 202 defined in the outer surface of at least a portion of the body. For example, the mold cavity may have an elongate cavity for the handle 108, a flat, disc-shaped cavity for the head 104, and a spiral protrusion in the disc-shaped cavity to form the spiral groove 102 in the planar front surface 112 of the head 104.

At 806, the hardened material is removed from the mold cavity. For example, the mold cavity may be opened and the hardened material may be removed from the opened mold cavity. The hardened material may be in the shape of the body. In some examples, at least a portion of the body may include a recess(es) 202 defined in an outer surface of the body.

At 808, the hardened material is subjected to surface treatment. The surface treatment promotes the capture of an analyte(s) found in a bodily fluid, such as saliva, when the oral sampling device is used for sample collection. In some examples, the entirety of the hardened material is surface treated at block 808. In other examples, a portion of the hardened material is surface treated, and a remainder of the hardened material is not surface treated. For example, the head 104 may be subjected to surface treatment at block 808 while the handle 108 is not subjected to the surface treatment. In some examples, the material of the body within the recess is the portion of the material that is subjected to surface treatment at block 808. As described herein, the surface treatment 808 applied to the hardened material at block 808 may include oxygen plasma treatment, coating the material with an affinity reagent(s), and/or hydrophilic treatment, among other surface treatments.

The process 800 describes an example injection molding process for manufacturing an oral sampling device, which may provide benefits of reproducibility and scaling up manufacturing to rapidly produce mass quantities of oral sampling devices. It is to be appreciated, however, that the oral sampling device described herein can be manufactured in other ways, such as three-dimensional (3D) printing, rapid prototyping or other additive manufacturing processes, or by cutting and/or milling (e.g., computer numerical control (CNC) milling) or other subtractive manufacturing processes. In one example of manufacturing the oral sampling device 100 using a milling technique, a polystyrene sheet having a thickness, T, is cut into a shape of the body (e.g., a body 106 having a disc-shaped head 104 and an elongate handle 108), and a recess(es) 202 (e.g., a spiral groove 102) is milled into the planar front surface 112 of the head 104 by CNC micromilling. In another example, a sheet of rigid porous material, such as porous polyethylene, porous polypropylene, or any other suitable rigid porous material (e.g., Porex®) having a thickness, T, is cut into a shape of the body.

FIG. 9 illustrates a flow diagram of an example process 900 for manufacturing an oral sampling device. For discussion purposes, the process 900 is described with reference to the previous figures. In some examples, the process 900 may continue from block 808 of the process 800.

At 902, ingredients for a flavored substance 300 are mixed and heated. In an example where the flavored substance 300 is flavored isomalt, block 902 may involve gradually adding about 1000 grams (g) of isomalt to about 80 g of boiling water until the solution reaches about 170° C., adding about 3.5 milliliters (mL) of colored gel (e.g., red gel color) after the solution reaches about 165° C., and adding about 10 mL of flavored candy oil after the solution reaches about 170° C. In some examples, the solution is removed from heat and cooled to room temperature and then remelted at block 904. In other examples, the solution is not cooled to room temperature between blocks 902 and 904.

At 904, the heated flavored substance 300 is poured into a mold. For mass manufacturing, multiple molds may be used and the heated flavored substance 300 is poured into the multiple molds. In some examples, the molds used at block 904 are made of food safe silicone (e.g., smooth-sil 940, Smooth-On, etc.), and the molds themselves may be made by pouring the food safe silicone into acrylic molds and cured at room temperature. In some examples, the mold(s) is/are placed onto a marble slab for rapid cooling of the heated flavored substance 300 when the heated flavored substance 300 is poured into the mold(s) at block 904.

At 906, the body of an oral sampling device, such as the oral sampling device manufactured using the process 800, is placed in the mold such that the recess(es) 202 (e.g., spiral groove 102) is facing up and the back surface 114 of the body 106 of the oral sampling device 100 is in contact with the flavored substance 300. The flavored substance 300 within the mold is allowed to harden while the body of the oral sampling device remains disposed in the mold and in contact with the flavored substance 300. In some examples, back surface 114 of the disc-shaped head 104 of the body 106 is set on the flavored substance 300 within the mold before the flavored substance 300 hardens, but after it has cooled to allow the head 104 to rest on the flavored substance 300 without the body 106 sinking into the flavored substance 300. In some examples, the body of the oral sampling device is held in place for a period of time (e.g., 15 seconds) until the flavored substance 300 starts to harden. In some examples, the body of the oral sampling device is pressed into the partially-hardened flavored substance 300 before it fully hardens. In some examples, the oral sampling device is set in the flavored substance 300 for a period of time (e.g., 5 to 10 minutes) to ensure that the flavored substance 300 is adhered to the body of the oral sampling device.

At 908, the oral sampling device with the flavored substance 300 disposed thereon is removed from the mold and packaged within a package. For example, the oral sampling device with the flavored substance 300 may be removed from the mold and placed on a silicone mat on a marble slab to finish cooling, and after cooling, the oral sampling device is placed into a polypropylene bag and heat sealed using an impulse sealer. The packaged oral sampling device may then be distributed to clinics and/or retailers (e.g., drug stores, grocery stores, etc.) and/or consumers.

In some examples, the manufactured oral sampling device may be packaged and sold to consumers, retailers, and/or to clinics as a kit (e.g., a testing kit, diagnostic kit, a pharmaceutical development kit, etc.) for disease diagnosis. For example, the kit may include the oral sampling device described herein. In some examples, the kit may include one or more additional components in addition to the oral sampling device. The one or more additional components in the kit may include an elution buffer and/or reagents to elute the analyte from the oral sampling device, PCR reagents, and/or protein detection reagents. The PCR reagents included in a kit may include a replication enzyme and deoxynucleotide and/or nucleotide triphosphates. The PCR reagents included in a kit may be specific to the analyte that is to be eluted from the oral sampling device. Such PCR reagents may include hybridizing primer sequences and probe sequences. The protein detection reagents included in a kit may include a protein binding domain linked to a detectable label, such as a fluorescent protein or bead. Such a protein binding domain may include an antibody or a binding fragment thereof, such as a single chain variable fragment (scFv). In some examples, the one or more additional components in the kit may include one or more tubes (e.g., 12 mL tubes) for eluting the analyte(s) from the oral sampling device, elution buffer, beads (e.g., glass beads) using in the elution methods described herein, and/or Micro centrifuge tubes (Micro tubes), or the like.

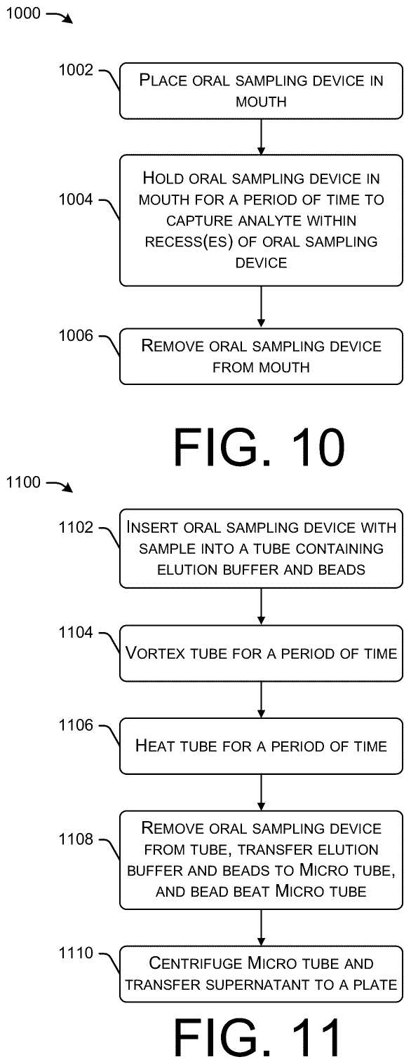

FIG. 10 illustrates a flow diagram of an example process 1000 for using an oral sampling device to collect a sample of bodily fluid, such as saliva. For discussion purposes, the process 1000 is described with reference to the previous figures.

At 1002, a human subject may place an oral sampling device in his or her mouth. In some examples, a portion of a body of the oral sampling device is placed in the mouth, such as by placing the head 104 of the oral sampling device 100 in the mouth at block 1002. In other examples, an entire body 606 of the oral sampling device 600 is placed in the mouth of the human subject at 1002.

At 1004, the human subject holds the oral sampling device (e.g., a portion thereof) in his or her mouth for a period of time to capture an analyte(s) 200 within the recess(es) 202 of the oral sampling device for evaluation of a condition in the human subject. The human subject may be informed of the period of time in various ways. In some examples, the sample collection period is provided to the human subject on instructions that come with the oral sampling device, or instructions that are verbally relayed to the human subject by a trained worker in a clinic (e.g., a nurse). Additionally, or alternatively, the flavored substance 300 (if included) may be configured to release a flavor over the period of time to indicate to the human subject that he or she should hold the oral sampling device in his or her mouth until the flavor ceases to be released by the flavored substance 300 (e.g., after the flavored substance 300 completely dissolves in the mouth).

At 1006, the human subject may remove the oral sampling device from his or her mouth. At this point, the recess(es) 202 (e.g., spiral groove 102) of the oral sampling device will have captured analytes 200. In a home setting, the human subject (or a caregiver of a child subject) may re-package the used oral sampling device and mail/ship it to a testing laboratory or schedule the oral sampling device for pickup at the subject's home. In a clinical setting, the human subject may give the oral sampling device back to a trained worker in the clinic (e.g., a nurse).

FIG. 11 illustrates a flow diagram of an example process 1100 for using an oral sampling device to test a sample that was collected by the oral sampling device. For discussion purposes, the process 1100 is described with reference to the previous figures.

At 1102, an oral sampling device that has been used to collect a sample of bodily fluid (e.g., saliva) from a human subject is inserted into a tube containing an elution buffer and beads (e.g., glass beads). For example, the oral sampling device 100 may be inserted, head 104 first, into a 12 mL tube (e.g., a polypropylene tube) containing about 300 microliters (μL) of elution buffer and about 100 μL of beads. As mentioned above, the size of the head (e.g., the head 104) of the oral sampling device may be suitable for inserting the head inside of a 12 mL tube, which is a common tube used in clinical chemistry laboratories. The length, L, of the oral sampling device may also be suitable for inserting the oral sampling device inside of such a tube. An example elution buffer is eSwab buffer containing 5% ethanol or 5% ethanol and 0.5% Triton. In some examples, the oral sampling device is initially tapped over a beaker containing 10% bleach to remove excess liquid from the recess(es) (e.g., spiral groove 102) of the oral sampling device before the oral sampling device is inserted into the tube containing the elution buffer and beads.

At 1104, the tube containing the oral sampling device is vortexed (e.g., subjected to translational and/or rotational movement) for a period of time. In an example, the vortex operation is performed at block 1104 for a period of about 50 seconds.

At 1106, the tube containing the oral sampling device is heated to an elevated temperature for a period of time. In an example, the elevated temperature is about 80° C. to about 85° C., and the period of time is about 10 minutes.

At 1108, the oral sampling device is removed from the tube, the elution buffer and the beads are transferred to a Micro centrifuge tube (Micro tube), and the Micro tube containing the elution buffer and the beads is bead beaten. Beat beating is a mechanical method used to disrupt a biological sample, and, in this case a sample of saliva containing an analyte(s) of interest. In an example, the Micro tube is a 2 mL polypropylene Micro tube, and the bead beating is performed twice for 30 seconds each time.

At 1110, the Micro tube is centrifuged and the resulting supernatant is transferred onto a plate for further analysis. In some examples, the tube is centrifuged at about 10000 G for about 30 seconds. The supernatant can be tested, such as by using a PCR test or a qPCR test, among other possible tests, to determine if a target analyte 200 is present in the sample. For example, a qPCR analysis can be conducted to detect the presence or absence of a target analyte 200, such as Streptococcus pyogenes. Although the process 1100 describes an example elution method, other elution methods or methods of extracting analyte(s) from the oral sampling device may be used, depending on the surface treatment used and/or the particular analyte(s) (e.g., pathogen) that is targeted.

The oral sampling device described herein may be used for collecting a sample from a human subject so that a condition in the human subject can be evaluated. Such a condition may be an infection, such as a viral infection, a bacterial infection, a fungal infection, and/or a yeast infection. In some examples, the condition may be influenza, tuberculosis, pneumonia, or strep throat. For example, the oral sampling device may be used to collect a sample to confirm the presence of Streptococcus pyogenes, which is indicative of streptococcal pharyngitis, such as GAS pharyngitis (also known as strep throat). Pharyngitis is a common reason for which children and young adults seek medical care, and GAS is the most frequent cause of bacterial pharyngitis in these age groups, and it can lead to suppurative complications (e.g., acute otitis media, sinusitis, retro- and peritonsillar abscess, cervical adenitis), rheumatic fever, and organ damage, if left undiagnosed and untreated. In some examples, a viral infection to be evaluated based on a sample collected by the oral sampling device is caused by a virus within the family Rhabdoviridae, Arenaviridae, Togaviridae, Filoviridae, Retroviridae, Coronaviridae, Paramyxoviridae, Flaviviridae, Orthomyxoviridae, and Baculoviridae. In particular embodiments, the analytes captured by the oral sampling device represent proteins, such as GPs derived from a genus including Vesiculovirus, Lyssavirus, Arenavirus, Alphavirus, Filovirus, Alpharetrovirus, Betaretrovirus, Gammaretrovirus, Deltaretrovirus, Spumavirus, Lentivirus, Coronavirus, Respirovirus, Hepacivirus, Influenzavirus A, or nucleopolyhedrovirus. In some examples, the viral infection is caused by coronavirus, respiratory syncytial virus (RSV), influenza virus, parainfluenza virus hepatitis virus, human immunodeficiency virus (HIV), measles virus, Dengue virus, Epstein Barr virus, or herpes simplex virus. In some examples, the coronavirus is Middle East respiratory syndrome coronavirus (MERS-CoV), severe acute respiratory syndrome coronavirus (SARS-CoV), or severe acute respiratory syndrome coronavirus 2 (SARS-CoV-2). In some examples, a bacterial infection to be evaluated based on a sample collected by the oral sampling device is caused by a bacterium within the family anthrax, gram-negative bacilli, chlamydia, diptheria, haemophilus influenza, Helicobacter pylori, Mycobacterium tuberculosis, pertussis toxin, pneumococcus, rickettsiae, staphylococcus, streptococcus and tetanus. In some examples, the bacterial infection is caused by pharyngitis, strep throat, tuberculosis, anthrax, tetanus, leptospirosis, pneumonia, cholera, botulism, pseudomonas infection, methicillin-resistant Staphylococcus aureus (MRSA) infection, Escherichia coli infection, Salmonella infection, Shigella infection, meningitis, gonorrhea, bubonic plague, syphilis, chlamydia, whooping cough, or Lyme disease. Accordingly, the disclosed oral sampling device is an effective sample collection instrument for diagnosing diseases.

In general, the sample collected by the oral sampling device can be tested using any suitable type of test. For example, a PCR test, a quantitative PCR (qPCR) test, a real-time PCR test, a multiplex PCR test, or the like can be used to determine the presence of an analyte(s) 200 of interest in the sample collected by the oral sampling device. For example, a pathogen panel, or a respiratory panel, may be used to test a number of different pathogens. Other ways of testing the collected sample include impedimetric biosensors, touch spray-mass spectrometry, and commercial test kits (e.g., BinaxNOW® Strep A Test; OSOM® Ultra Strep A Test; BD Chek™ Group A Strep; QuickVue In-line Strep A Test).

An example PCR test is Droplet Digital™ PCR (ddPCR™). Particular embodiments utilize ddPCR™ (Bio-Rad Laboratories, Hercules, CA). ddPCR technology uses a combination of microfluidics and surfactant chemistry to divide PCR samples into water-in-oil droplets. Hindson et al., Anal. Chem. 83(22): 8604-8610 (2011). The droplets support PCR amplification of the target template molecules they contain and use reagents and workflows similar to those used for most standard Taqman probe-based assays. For example, within the context of the current disclosure, a representative droplet would include fragmented RNA or DNA for analysis and hybridizing primer/probe sequences.

Following PCR, each droplet is analyzed or read in a flow cytometer to determine the fraction of PCR-positive droplets in the original sample. These data are then analyzed using Poisson statistics to determine the target concentration in the original sample. See Bio-Rad Droplet Digital™ (ddPCR™) PCR Technology.

While ddPCR™ is a preferred approach, other sample partition PCR methods based on the same underlying principles may also be used. These approaches are now described more generally.

Sample Partitioning. Numerous methods can be used to divide samples into discrete partitions (e.g., droplets). Exemplary partitioning methods and systems include use of one or more of emulsification, droplet actuation, microfluidics platforms, continuous-flow microfluidics, reagent immobilization, and combinations thereof. In some embodiments, partitioning is performed to divide a sample into a sufficient number of partitions such that each partition contains one or zero nucleic acid molecules. In some embodiments, the number and size of partitions is based on the concentration and volume of the bulk sample.

Methods and devices for partitioning a bulk volume into partitions by emulsification are described in Nakano et al. J. Biotechnol. 102, 117-124 (2003) and Margulies et al. Nature 437, 376-380 (2005). Systems and methods to generate “water-in-oil” droplets are described in U.S. Publication No. 2010/0173394. Microfluidics systems and methods to divide a bulk volume into partitions are described in U.S. Publication Nos. 2010/0236929; 2010/0311599; and 2010/0163412, and U.S. Pat. No. 7,851,184. Microfluidic systems and methods that generate monodisperse droplets are described in Kiss et al. Anal. Chem. 80(23), 8975-8981 (2008). Further microfluidics systems and methods for manipulating and/or partitioning samples using channels, valves, pumps, etc. are described in U.S. Pat. No. 7,842,248. Continuous-flow microfluidics systems and methods are described in Kopp et al., Science, 280, 1046-1048 (1998).

Partitioning methods can be augmented with droplet manipulation techniques, including electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In some embodiments, a droplet microactuator is supplemented with a microfluidics platform (e.g. continuous flow components).

Some embodiments use a droplet microactuator. A droplet microactuator can be capable of effecting droplet manipulation and/or operations, such as dispensing, splitting, transporting, merging, mixing, agitating, and the like. Droplet operation structures and manipulation techniques are described in U.S. Publication Nos. 2006/0194331 and 2006/0254933 and U.S. Pat. Nos. 6,911,132; 6,773,566; and 6,565,727.

Amplification. The partitioned nucleic acids of a sample can be amplified by any suitable PCR methodology that can be practiced with PCR assays. Exemplary PCR types include allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, single-cell PCR, solid-phase PCR, thermal asymmetric interlaced PCR, touchdown PCR, universal fast walking PCR, etc. Ligase chain reaction (LCR) may also be used.

PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others.

PCR are driven by thermal cycling. Alternative amplification reactions, which may be performed isothermally, can also be used. Exemplary isothermal techniques include branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, etc.

Amplification may be performed with any suitable reagents (e.g. template nucleic acid (e.g. DNA or RNA)), primers, probes, buffers, replication catalyzing enzymes (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase), and deoxynucleotide (and/or nucleotide) triphosphates (dNTPs and/or NTPs), etc.

Amplification reagents can be added to a sample prior to partitioning, concurrently with partitioning and/or after partitioning has occurred. In some embodiments, all partitions are subjected to amplification conditions (e.g. reagents and thermal cycling), but amplification only occurs in partitions containing target nucleic acids (e.g. nucleic acids containing sequences complementary to primers added to the sample). The template nucleic acid can be the limiting reagent in a partitioned amplification reaction. In some embodiments, a partition contains one or zero target (e.g. template) nucleic acid molecules.

As indicated previously, in some embodiments, nucleic acid targets, primers, and/or probes are immobilized to a surface, for example, a substrate, plate, array, bead, particle, etc. Immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In some embodiments, assay reagents and/or target nucleic acids are immobilized to a surface while retaining the capability to interact and/or react with other reagents (e.g. reagent dispensed from a microfluidic platform, a droplet microactuator, etc.). In some embodiments, reagents are immobilized on a substrate and droplets or partitioned reagents are brought into contact with the immobilized reagents. Techniques for immobilization of nucleic acids and other reagents to surfaces are well understood by those of ordinary in the art. See, for example, U.S. Pat. No. 5,472,881 and Taira et al. Biotechnol. Bioeng. 89(7), 835-8 (2005).

Target Sequence Detection. Detection methods can be utilized to identify sample partitions containing amplified target(s). Detection can be based on one or more characteristics of a sample partition such as a physical, chemical, luminescent, or electrical aspects, which correlate with amplification.

In particular embodiments, fluorescence detection methods are used to detect amplified target(s), and/or identification of sample partitions containing amplified target(s). Exemplary fluorescent detection reagents include TaqMan probes, SYBR Green fluorescent probes, molecular beacon probes, scorpion probes, and/or LightUp Probes® (LightUp Technologies AB, Huddinge, Sweden). Additional detection reagents and methods are described in, for example, U.S. Pat. Nos. 5,945,283; 5,210,015; 5,538,848; and 5,863,736; PCT Publication WO 97/22719; and publications: Gibson et al., Genome Research, 6, 995-1001 (1996); Heid et al., Genome Research, 6, 986-994 (1996); Holland et al., Proc. Natl. Acad. Sci. USA 88, 7276-7280, (1991); Livak et al., Genome Research, 4, 357-362 (1995); Piatek et al., Nat. Biotechnol. 16, 359-63 (1998); Neri et al., Advances in Nucleic Acid and Protein Analysis, 3826, 117-125 (2000); Compton, Nature 350, 91-92 (1991); Thelwell et al., Nucleic Acids Research, 28, 3752-3761 (2000); Tyagi and Kramer, Nat. Biotechnol. 14, 303-308 (1996); Tyagi et al., Nat. Biotechnol. 16, 49-53 (1998); and Sohn et al., Proc. Natl. Acad. Sci. U.S.A. 97, 10687-10690 (2000).

In some embodiments, detection reagents are included with amplification reagents added to the bulk or partitioned sample. In some embodiments, amplification reagents also serve as detection reagents. In some embodiments, detection reagents are added to partitions following amplification. In some embodiments, measurements of the absolute copy number and the relative proportion of target nucleic acids in a sample (e.g. relative to other targets nucleic acids, relative to non-target nucleic acids, relative to total nucleic acids, etc.) can be measured based on the detection of sample partitions containing amplified targets.

In some embodiments, following amplification, sample partitions containing amplified target(s) are sorted from sample partitions not containing amplified targets or from sample partitions containing other amplified target(s). In some embodiments, sample partitions are sorted following amplification based on physical, chemical, and/or optical characteristics of the sample partition, the nucleic acids therein (e.g. concentration), and/or status of detection reagents. In some embodiments, individual sample partitions are isolated for subsequent manipulation, processing, and/or analysis of the amplified target(s) therein. In some embodiments, sample partitions containing similar characteristics (e.g. same fluorescent labels, similar nucleic acid concentrations, etc.) are grouped (e.g. into packets) for subsequent manipulation, processing, and/or analysis.

Molecules that bind proteins when the analyte is a protein are described elsewhere herein as capture reagents. Such molecules can similarly be used as detection reagents.

Kits. Kits disclosed herein include materials to assay a sample for a biological marker. In particular embodiments, the kits include an oral sampling device as described herein. Kits cdan additionally include materials to detect nucleic acids, proteins, or the biological analytes. Materials to conduct PCR include components of amplification mixtures, such as at least one primer or primer pair, at least one probe, at least one replication enzyme (e.g., at least one polymerase), and deoxynucleotide (and/or nucleotide) triphosphates (dNTPs and/or NTPs), etc. Particular kits include protein binding molecules that bind a biomarker protein. Protein binding molecules are described elsewhere herein.

Additional embodiments include detection reagents. Exemplary detection reagents can include radioactive isotopes or radiolabels (e.g., 32P and 13C), enzymes (e.g., luciferase, HRP and AP), dyes (e.g., rhodamine and cyanine), fluorescent tags or dyes (e.g., GFP, YFP, FITC), magnetic beads, or biotin. In particular embodiments, the detectable label is fluorescein, GFP, rhodamine, cyanine dyes, Alexa dyes, luciferase, or a radiolabels. TaqMan probes, SYBR Green fluorescent probes, molecular beacon probes, scorpion probes, and/or LightUp Probes® (LightUp Technologies) may also be used.

Particular embodiments can include reference levels and/or control conditions (positive and/or negative).

Instructions for carrying out and interpreting assays, including, optionally, instructions for generating a score, can also be included in a kit. Instructions can be provided in written, taped, videoed, VCR, CD-ROM, flashdrive, USB formats or can be provided on a website or other remote location.

In particular embodiments, kits exclude equipment (e.g., plate readers). In particular embodiments, kits exclude materials commonly found in laboratory settings (pipettes; test tubes; distilled H2O).

### EXAMPLE TESTING

Testing has been performed on prototype oral sampling devices with designs that conform to the descriptions in the previous section to demonstrate the ability to capture Streptococcus pyogenes and control sample timing using flavor release. A few examples are provided below.

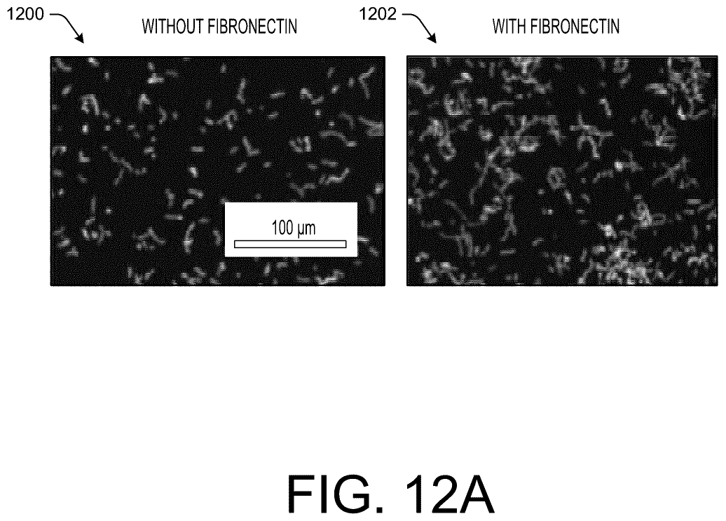

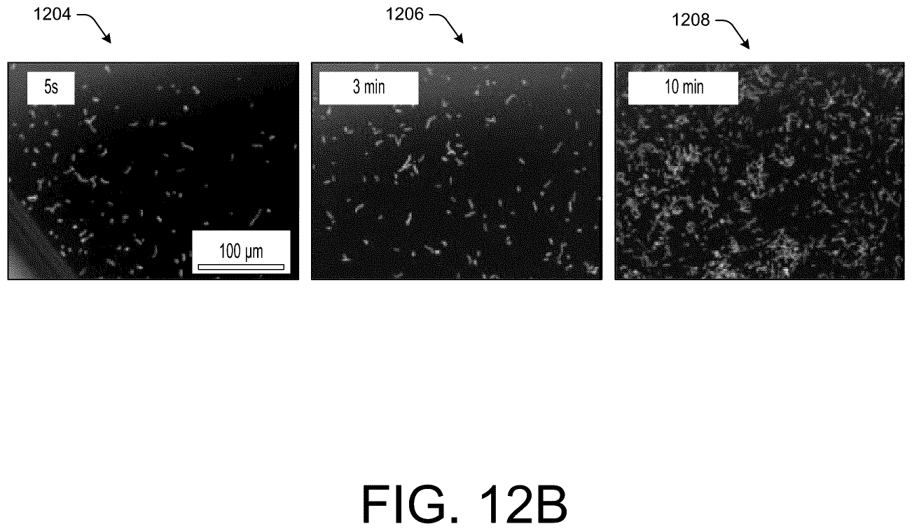

Example 1: A fibronectin coated surface was selected as a proof of concept for surface-treating the material of the body of the oral sampling device to capture Streptococcus pyogenes within the recess(es) 202 of the oral sampling device. FIG. 12A illustrates that coating a material of the body of the oral sampling device within the recess(es) 202 with fibronectin improves capture of Streptococcus pyogenes. Fibronectin functionalization-dependent measurements were carried out in the presence of both fibronectin and GAS (image 1202), or the absence of fibronectin in the presence of GAS (image 1200). Captured Streptococcus pyogenes was represented by fluorescence using Alexa Fluor 488 conjugated with wheat germ agglutinin (WGA), a common Streptococcus pyogenes labelling method. The results show that fibronectin functionalization enhances the capture of Streptococcus pyogenes on polystyrene surfaces.

Example 2: Different diseases have different densities of the pathogens in saliva. FIG. 12B illustrates that the amount of Streptococcus pyogenes captured using a fibronectin-coated polystyrene body of the oral sampling device increases with time. FIG. 12B shows that the amount of Streptococcus pyogenes captured on the surface of fibronectin-coated polystyrene can accrue with sampling time. Streptococcus pyogenes binding assay was carried out using a fibronectin concentration of 5 microgram per centimeter squared (μg/cm²) and three different sampling time periods (5 seconds, 3 minutes, and 10 minutes) with Streptococcus pyogenes concentration of 1×10⁷ colony forming unit (CFU)/mL, chosen as an upper concentration of Streptococcus pyogenes that can be cultured in human saliva. Briefly, Streptococcus pyogenes was suspended in stock WGA and Todd-Hewitt broth (THY) broth solutions. The resulting solution was added to each well in a 96-well plate and left for the three specified time periods, representing the sampling time. Streptococcus pyogenes suspension was pipetted out from the 96-well and the well was washed once with phosphate buffered saline (PBS), followed by fixing with 4% PFA. It was observed that of the amount of Streptococcus pyogenes captured on a fibronectin-coated surface increases over time, as evidenced by the image 1204 taken at 5 seconds, the image 1206 taken at 3 minutes, and the image 1208 taken at 10 minutes, corresponding to the three different sampling time periods. The ability to accumulate pathogens/bacteria over time can enable the tailoring the oral sampling device to the pathogen that is being tested based on its expected concentration in saliva.

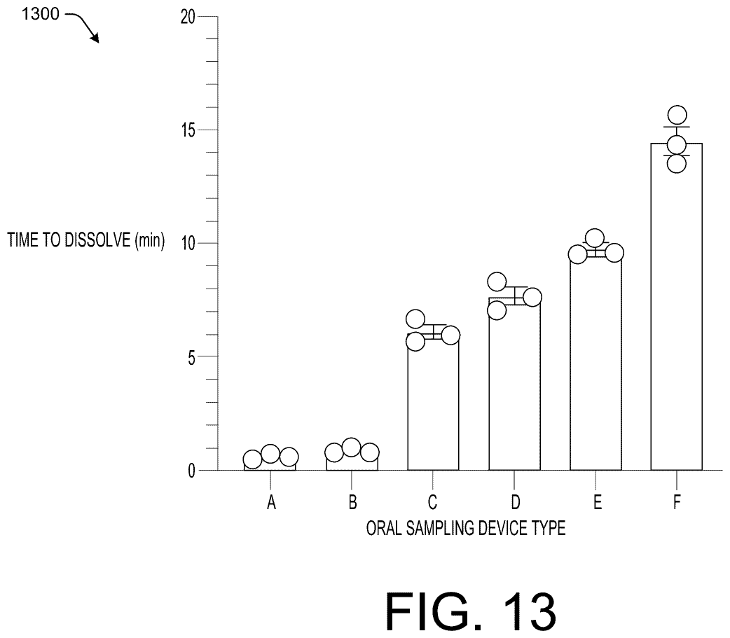

Example 3: As noted above, the flavored substance 300 may be used as a built-in, analog timer to communicate the sample collection period to the human subject without relying on the human subject to follow written instructions. Controlled amounts of flavoring and sugar alcohol isomalt (a common ingredient in “sugar-free” candy) were integrated into the oral sampling device to both control the sampling time and increase children's compliance. Since the surface area-to-mass ratio of the isomalt on the oral sampling device affects the dissolving time, specific dissolving times can be targeted based on the time period to capture pathogens of interest in vivo. Prototype oral sampling devices of varying sizes and surface area-to-mass ratios were designed and consumed to determine the dissolving time. FIG. 13 illustrates a graph 1300 showing dissolving periods of flavoring substance of six different types of oral sampling devices. Data in FIG. 13 are from one human subject. A time-to-dissolve range from 30 seconds to 15 minutes was achieved with several oral sampling device types, and decreasing or increasing the mass and/or surface area-to-mass ratio of the flavored substance 300 on the oral sampling device can expand the time-to-dissolve range.

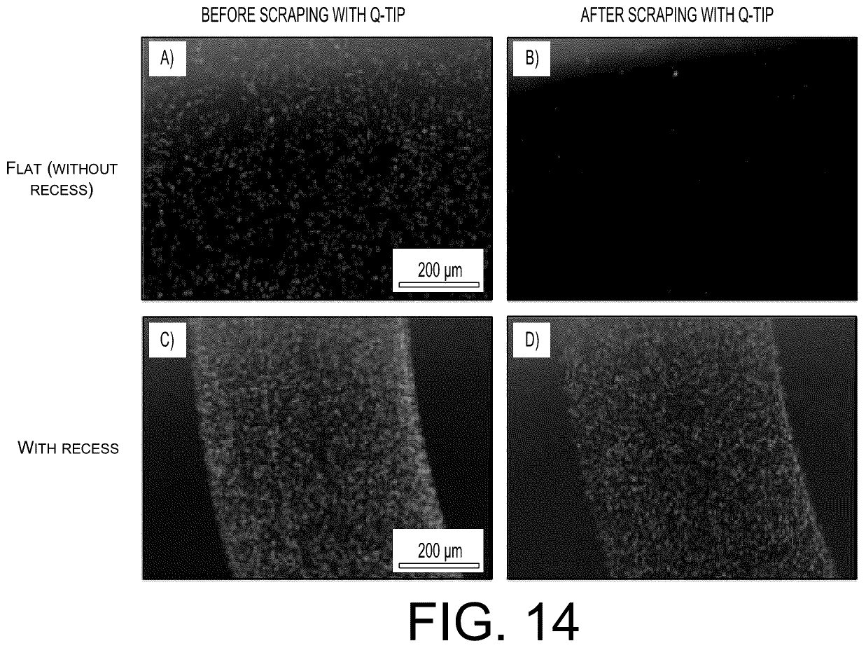

Example 4: An open recess(es) 202 is defined in the outer surface of the body of the oral sampling device to allow for functionalization, access for pathogen capture, and protection of the pathogens once captured. The geometry of the recess(es) 202 helps to prevent loss of captured pathogens due to mechanical friction encountered in the mouth during sample collection. In this friction test, the respective surfaces of two models of the oral sampling device were gently scraped with a cotton-tipped swab to simulate interaction with the tongue. FIG. 14 illustrates that the geometry of the recess 202 defined in the outer surface of the body of the oral device can mitigate loss of captured analytes due to mechanical friction. For example, images of the fluorescently-tagged bacteria were taken before and after the friction test was applied on the two models of the oral sampling device. Images A and B shown in FIG. 14 show before and after images, respectively, of the friction test applied to a simplified model of the oral sampling device, where the polystyrene surface is flat and fibronectin is coated on the surface. Images C and D shown in FIG. 14 show before and after images, respectively, of the friction test applied to a model in which a recess 202 was milled into the polystyrene surface and fibronectin was coated exclusively on the surface of the recess. While in the flat oral sampling device model (images A and B) a decrease in bacteria on the surface was observed following the application of mechanical friction, bacteria were not lost from the oral sampling device model (images C and D) with the recess 202 after mechanical friction (e.g., a substantial portion of the bacteria remain within the recess 202 after gentle scraping with a cotton-tipped swab), thereby validating that the geometry of the recess 202 can mitigate loss of captured analytes, such as bound bacteria, during mechanical stress.

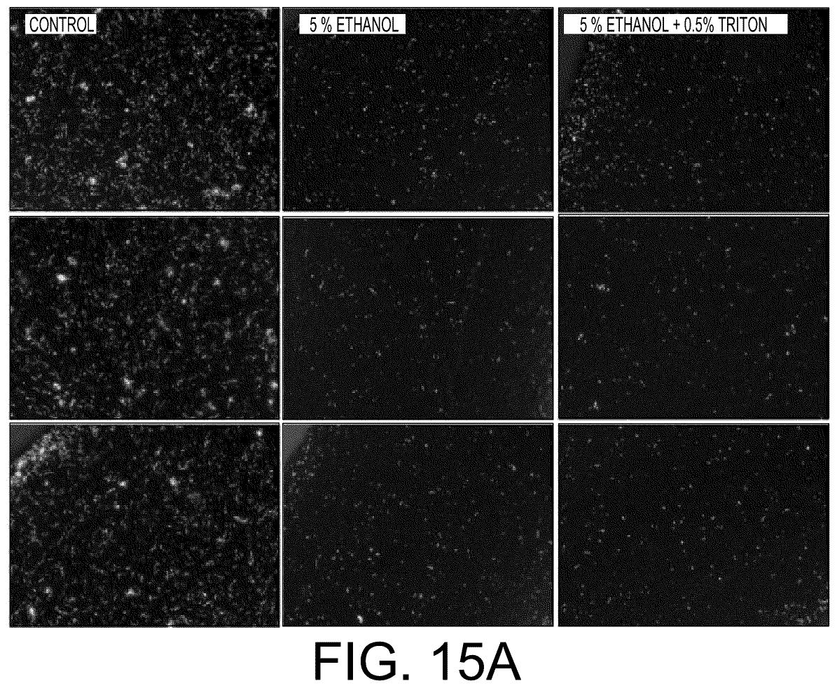

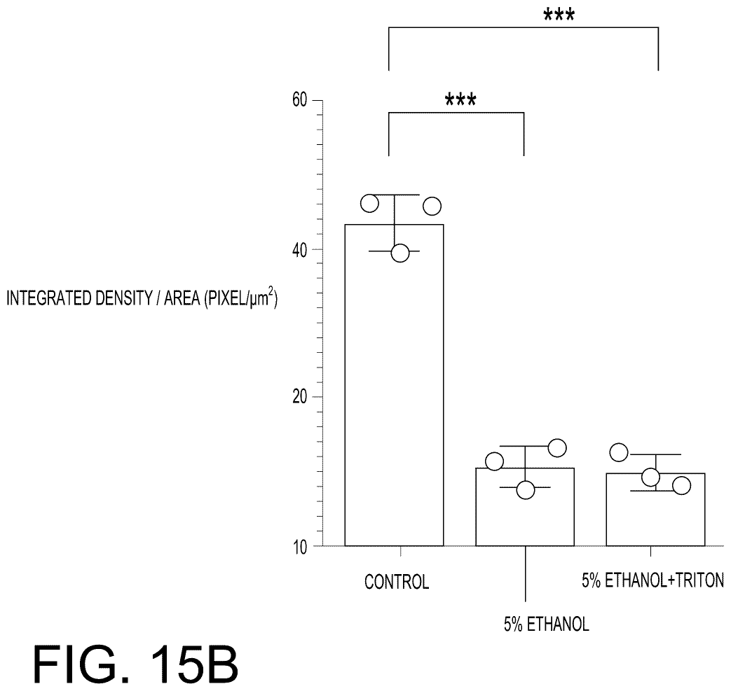

Example 5: In FIG. 15A, Streptococcus mutans (10¹ CFU/mL) was used to verify the of elution methods described herein, such as the process 1100 of FIG. 11. The bacteria on the oral sampling device were stained and imaged on an oral sampling device surface-treated with oxygen plasma treatment. Note that the elution method may be different for different target pathogens or surface treatments. For example, surface treatments, such as antibodies, proteins or aptamers, may require different elution methods. Streptococcus mutans with 109 CFU/mL concentration was used to visualize the effect of elution method. The leftmost column (labeled “control” in FIG. 15A) shows the control (without any elution), the middle column (labeled “5% Ethanol in FIG. 15A) shows elution with eSwab buffer with 5% Ethanol, and the rightmost column (labeled “5% Ethanol +0.5% Triton) shows elution with eSwab buffer with 5% Ethanol+0.5% Triton. This demonstrates the efficacy of the elution methods described herein. FIG. 15B illustrates quantitative data to demonstrate the efficacy of the elution method described herein. The quantitative data represents the effect of elution methods in integrated density/area (pixel/μm²) showed that both elution methods has significantly removed Streptococcus mutans on the oral sampling device compared to the control.

### EXAMPLE CLAUSES

1. An oral sampling device comprising: a body, wherein at least a portion of the body: is sized to fit inside of a mouth of a human; and comprises: an outer surface; and a recess defined in the outer surface, a material of the body within the recess having been subjected to a surface treatment to promote capture of an analyte found in saliva.
2. The oral sampling device of clause 1, wherein the recess comprises at least one of a groove or a pit.
3. The oral sampling device of clause 2, wherein: the groove is at least one of: a spiral groove having more than one turn; or one or more grooves having a radial pattern; or the pit is one of multiple pits spatially distributed across the outer surface.
4. The oral sampling device of any one of clauses 1 to 3, wherein the surface treatment comprises at least one of: oxygen plasma treatment; coating the material with an affinity reagent; or hydrophilic treatment.
5. The oral sampling device of clause 4, wherein the affinity reagent is fibronectin or collagen.
6. The oral sampling device of clause 4, wherein the affinity reagent is an aptamer.
7. The oral sampling device of clause 4, wherein the affinity reagent is a binding molecule for the analyte.
8. The oral sampling device of clause 7, wherein the binding molecule is a nucleic acid that hybridizes to the analyte or is a protein binding domain that binds the analyte.
9. The oral sampling device of clause 8, wherein the protein binding domain comprises an antibody or a binding fragment thereof.
10. The oral sampling device of clause 9, wherein the binding fragment thereof is a single chain variable fragment (scFv).
11. The oral sampling device of any one of clauses 1 to 10, wherein the recess has a depth equal to or greater than 0.5 millimeter.
12. The oral sampling device of any one of clauses 1 to 11, wherein the recess has a width equal to or less than 2 millimeters.
13. The oral sampling device of any one of clauses 1 to 12, further comprising a flavored substance disposed on at least the portion of the body, wherein the flavored substance does not cover the recess.
14. The oral sampling device of clause 13, wherein the flavored substance is configured to release a flavor over a period of time while at least the portion of the body is disposed inside of the mouth, the period of time substantially corresponding to a target amount of time the oral sampling device is to remain inside of the mouth.
15. The oral sampling device of clause 13, wherein the flavored substance is at least one of a gel, a hard candy, or a gummy material.
16. The oral sampling device of any one of clauses 1 to 15, wherein the body comprises a handle and a head coupled to the handle, wherein at least the portion of the body comprises the head.
17. A method of using the oral sampling device of any one of clauses 1 to 16, the method comprising capturing the analyte within the recess for evaluation of a condition in the human.
18. The method of clause 17, wherein the condition is an infection.
19. The method of clause 18, wherein the infection is a viral infection, a bacterial infection, a fungal infection, or a yeast infection.
20. The method of clause 19, wherein the viral infection is caused by a virus within the family Rhabdoviridae, Arenaviridae, Togaviridae, Filoviridae, Retroviridae, Coronaviridae, Paramyxoviridae, Flaviviridae, Orthomyxoviridae, and Baculoviridae.
21. The method of clause 19, wherein the viral infection is caused by coronavirus, respiratory syncytial virus (RSV), influenza virus, parainfluenza virus hepatitis virus, human immunodeficiency virus (HIV), measles virus, Dengue virus, Epstein Barr virus, or herpes simplex virus.
22. The method of clause 21, wherein the coronavirus is Middle East respiratory syndrome coronavirus (MERS-CoV), severe acute respiratory syndrome coronavirus (SARS-CoV), or severe acute respiratory syndrome coronavirus 2 (SARS-CoV-2).
23. The method of clause 19, wherein the bacterial infection is caused by a bacterium within the family anthrax, gram-negative bacilli, chlamydia, diptheria, haemophilus influenza, Helicobacter pylori, Mycobacterium tuberculosis, pertussis toxin, pneumococcus, rickettsiae, staphylococcus, streptococcus and tetanus.
24. A kit comprising the oral sampling device of any one of clauses 1 to 16.
25. The kit of clause 24, further comprising reagents to elute the analyte from the oral sampling device.
26. The kit of clause 24, further comprising polymerase chain reaction (PCR) reagents.
27. The kit of clause 26, wherein the PCR reagents are specific to the analyte.
28. The kit of clause 27, wherein the PCR reagents specific for the analyte comprise hybridizing primer sequences and probe sequences.
29. The kit of clause 26, wherein the PCR reagents comprise a replication enzyme and deoxynucleotide and/or nucleotide triphosphates.
30. The kit of clause 24, further comprising protein detection reagents.
31. The kit of clause 30, wherein the protein detection reagents comprise a protein binding domain linked to a detectable label.
32. The kit of clause 31, wherein the protein binding domain comprises an antibody or a binding fragment thereof.
33. The kit of clause 32, wherein the binding fragment thereof is a single chain variable fragment (scFv).
34. The kit of clause 31, wherein the detectable label is a fluorescent protein or bead.
35. An oral sampling device comprising: a body, wherein at least a portion of the body: is sized to fit inside of a mouth of a human; and comprises: an outer surface; and a recess defined in the outer surface; and a flavored substance disposed on at least the portion of the body, wherein the flavored substance: does not cover the recess; and is configured to release a flavor over a period of time while at least the portion of the body is disposed inside of the mouth.
36. The oral sampling device of clause 35, wherein the period of time corresponds to a target amount of time the oral sampling device is to remain inside of the mouth.
37. The oral sampling device of clause 35 or 36, wherein the flavored substance is configured to dissolve over the period of time while at least the portion of the body is disposed inside of the mouth.
38. The oral sampling device of any one of clauses 35 to 37, wherein the flavored substance is configured to cease releasing the flavor after at least the portion of the body has been disposed inside of the mouth for the period of time.
39. The oral sampling device of any one of clauses 35 to 38, wherein the flavored substance is configured to change color after at least the portion of the body has been disposed inside of the mouth for the period of time.
40. The oral sampling device of any one of clauses 35 to 39, wherein the flavored substance is an artificial sweetener.
41. The oral sampling device of clause 40, wherein the artificial sweetener is flavored isomalt.
42. The oral sampling device of any one of clauses 35 to 41, wherein the flavored substance is configured to stimulate a neurological response that causes the human to produce an analyte found in saliva.
43. The oral sampling device of any one of clauses 35 to 42, wherein a material of the body within the recess has been subjected to a surface treatment to promote capture of an analyte found in saliva.
44. An oral sampling device comprising: a body, wherein at least a portion of the body is: sized to fit inside of a mouth of a human; and made of a rigid porous material; and a flavored substance disposed on at least the portion of the body, wherein the flavored substance:
does not cover at least some of rigid porous material; and is configured to release a flavor over a period of time while at least the portion of the body is disposed inside of the mouth.
   45. The oral sampling device of clause 43, wherein the body comprises a handle and a head coupled to the handle, wherein at least the portion of the body comprises the head.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; 18% of the stated value; 17% of the stated value; 16% of the stated value; 15% of the stated value; 14% of the stated value; 13% of the stated value; 12% of the stated value; 11% of the stated value; 10% of the stated value; 9% of the stated value; 8% of the stated value; ±7% of the stated value; ±6% of the stated value; 5% of the stated value; ±4% of the stated value; ±3% of the stated value; ±2% of the stated value; or +1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. An oral sampling device comprising:
a body, wherein at least a portion of the body:
is sized to fit inside of a mouth of a human; and
comprises:
an outer surface; and
a recess defined in the outer surface,
a material of the body within the recess having been subjected to a surface treatment to promote capture of an analyte found in saliva.

2. The oral sampling device of claim 1, wherein the recess comprises at least one of a groove or a pit.

3. The oral sampling device of claim 2, wherein:
the groove is at least one of:
a spiral groove having more than one turn; or
one or more grooves having a radial pattern; or
the pit is one of multiple pits spatially distributed across the outer surface.

4. The oral sampling device of claim 1, wherein the surface treatment comprises at least one of:
oxygen plasma treatment;
coating the material with an affinity reagent; or
hydrophilic treatment.

5. The oral sampling device of claim 4, wherein the affinity reagent is fibronectin or collagen.

6. The oral sampling device of claim 4, wherein the affinity reagent is an aptamer.

7. The oral sampling device of claim 4, wherein the affinity reagent is a binding molecule for the analyte.

8. The oral sampling device of claim 7, wherein the binding molecule is a nucleic acid that hybridizes to the analyte or is a protein binding domain that binds the analyte.

9. The oral sampling device of claim 8, wherein the protein binding domain comprises an antibody or a binding fragment thereof.

10. The oral sampling device of claim 9, wherein the binding fragment thereof is a single chain variable fragment (scFv).

11. The oral sampling device of claim 1, wherein the recess has a depth equal to or greater than 0.5 millimeter.

12. The oral sampling device of claim 1, wherein the recess has a width equal to or less than 2 millimeters.

13. The oral sampling device of claim 1, further comprising a flavored substance disposed on at least the portion of the body, wherein the flavored substance does not cover the recess.

14. The oral sampling device of claim 13, wherein the flavored substance is configured to release a flavor over a period of time while at least the portion of the body is disposed inside of the mouth.

15. The oral sampling device of claim 13, wherein the flavored substance is at least one of a gel, a hard candy, a gummy material, an artificial sweetener, or flavored isomalt.

16. The oral sampling device of claim 1, wherein the body comprises a handle and a head coupled to the handle, wherein at least the portion of the body comprises the head.

17-36. (canceled)

37. The oral sampling device of claim 14, wherein the flavored substance is configured to dissolve over the period of time while at least the portion of the body is disposed inside of the mouth.

38. The oral sampling device of claim 14, wherein the flavored substance is configured to cease releasing the flavor after at least the portion of the body has been disposed inside of the mouth for the period of time.

39. The oral sampling device of claim 14, wherein the flavored substance is configured to change color after at least the portion of the body has been disposed inside of the mouth for the period of time.

40. (canceled)

41. (canceled)

42. The oral sampling device of claim 13, wherein the flavored substance is configured to stimulate a neurological response that causes the human to produce the analyte.

43-45. (canceled)
